(19) United States
(12) Patent Application Publication
(10) Pub. No.: US 2024/0424147 A1
(43) Pub. Date: Dec. 26, 2024

(54) PHTHALOCYANINE-LOADED MICELLES FOR THE DIRECT VISUALIZATION OF TUMORS

(71) Applicant: THE TRUSTEES OF THE UNIVERSITY OF PENNSYLVANIA, Philadelphia, PA (US)

(72) Inventors: Andrew TSOURKAS, Bryn Mawr, PA (US); Ahmad Amirshaghaghi, Philadelphia, PA (US); ZHILIANG CHENG, Newtown, PA (US); Yiran TIAN, Haddon Township, NJ (US); Karthik SUNDARAM, Media, PA (US)

(73) Assignee: THE TRUSTEES OF THE UNIVERSITY OF PENNSYLVANIA, Philadelphia, PA (US)

(21) Appl. No.: 18/698,777

(22) PCT Filed: Oct. 5, 2022
(86) PCT No.: PCT/US2022/077633

§ 371 Date: Apr. 4, 2024

(51) Int. Cl. A61K 49/00

(52) U.S. Cl. CPC A61K 49/0032; A61K 49/0052; A61K 49/006

(57) ABSTRACT

Provided are compositions comprising phthalocyanine (PC)-loaded or naphthalocyanine (NC) dye-loaded nanoparticulate polymeric or lipid micelles and methods for visualization of tumors without optical imaging equipment, the methods comprising injecting a subject with the phthalocyanine (PC)-loaded nanoparticulate amphiphilic polymer or lipid micelles and b) detecting by endoscopy an accumulation of blue dye in a tumor and a demarcation of tumor margins. Also provided are methods for treating a tumor, the method comprising a) administering intravenously to a subject phthalocyanine (PC) dye-loaded or naphthalocyanine (NC) dye-loaded nanoparticulate amphiphilic polymer or lipid micelles; b) detecting by intraoperative photoacoustic imaging an accumulation of dye in a tumor and a demarcation of tumor margins; and/or c) irradiating the PC dye or the NC dye within the tumor with a laser to heat the tumor, thereby decreasing tumor cell viability and/or killing the tumor cells.

### STATEMENT REGARDING FEDERALLY SPONSORED RESEARCH

This invention was made with government support under EB029238, EB028858 and TR001878 awarded by the National Institutes of Health. The government has certain rights in the invention.

### FIELD OF THE INVENTION

The present disclosure relates to compositions comprising phthalocyanine (PC)-loaded nanoparticulate polymeric or lipid micelles and methods for visualization tumors without optical imaging equipment, the methods comprising injecting a subject with the phthalocyanine (PC)-loaded nanoparticulate amphiphilic polymer or lipid micelles and b) detecting by endoscopy an accumulation of blue dye in a tumor and a demarcation of tumor margins.

### BACKGROUND OF THE INVENTION

Colorectal cancer (CRC) is one of the most common types of cancer in the world, ranks third and second in incidence and mortality respectively among the types of cancer cases, with more than 1.2 million new cases diagnosed annually. As most CRC cases show the transformation of adenomas into carcinomas over time, early detection and removal of colorectal adenomas are vital for its prevention. The reasons that adenomas or cancers are missed is thought to be associated with the location of the lesions or the skills of the endoscopist, e.g., the inability to differentiate them from healthy tissue. Conventional white-light endoscopy (WLE) plays a vital role in detecting and removing lesions of the digestive tract. However, detecting early gastrointestinal tract malignancy can be challenging with WLE, especially as dysplastic lesions are subtle and inconspicuous, and thus not visible to the naked eye using conventional WLE equipment. A substantial miss rate (11%-27%) has been reported for WLE detection of (pre) malignant colon lesions, particularly in high-risk patients, which compromises early detection and intervention.

Dye-based chromoendoscopy (DBC) has been introduced to improve patient outcomes, enabling the detection of dysplastic lesions in long-standing inflammatory bowel diseases (IBD). DBC involves applying mucosal staining or water-soluble blue dyes (e.g., carmine or methylene blue), usually by injection down an endoscopic spray-catheter. However, DBC has several limitations that hamper the feasibility of using DBC in daily routine clinical practice. First, it is a time-consuming procedure, thus it is currently not recommended for average-risk subjects, and the dye is not always evenly distributed through the surface, so distinguishing between cancerous, e.g., flat and/or polyp, and noncancerous tissue during resection is challenging, and the rate of missing tumor cells is high. Furthermore, most gastroenterologists do not follow the DBC biopsy protocol frequently during surgery, as it is a time—and cost-expensive approach. Meanwhile, for intraoperative navigation, the surgeon has to rely primarily on visual and tactile feedback to distinguish between different kinds of tissue structures. Together with the increasing rate of minimally invasive, such as endoscopic/laparoscopic and robotic surgery and, therefore, the lack of tactile feedback, there is a demand for improving the visibility of different kinds of tissue types, especially for distinguishing malignant and benign structures. However, as mentioned above, current classical stains or dyes, i.e., small molecules, lack sufficient tumor selectivity and are also taken up in healthy tissue, resulting in poor visibility of CRCs. Recently, a method of delivering contrast enhancement to the colon was developed using methylene-blue multimatrix (MB-MMX) to facilitate colonic lumen delivery, i.e., maximize staining intensity, and overcome the limitations of colonoscopic dye spray. But, efficacy and safety, e.g., risk of DNA damage, of MB-MMX remains a concern. Accordingly, there remains a clear unmet need for sensitive compositions and methods that enable detection of flat polyps in the CRC, ideally at a premalignant stage before invasive cancers develop.

Among women, ovarian cancer is the fifth most common cause of death due to cancer, and it is the deadliest of all the gynecological cancers. Due to the lack of early screening and diagnostic techniques, many women are diagnosed with ovarian cancer when it is already at stages III or IV, where the mortality rates are high (70 to 75%). Moreover, the high mortality rate among patients with metastatic ovarian cancer is attributed to the fact that only surgical removal of most of its abdominal metastases may reduce cancer recurrence and enhance the effect of postoperative chemotherapy. Unfortunately, even with the best microsurgical techniques, resection leaves behind residual microscopic tumors, which eventually lead to cancer relapse. Furthermore, the effect of postoperative chemotherapy is significantly compromised by the resistance of ovarian cancer cells to chemotherapeutic agents and the serious side effects that nontargeted chemotherapeutic agents have on healthy organs. Thus, there remains a need for developing compositions and methods to directly visualize tumors and improve complete tumor resection, the identification of metastases, and the treatment of any residual disease during surgery, bis(trihexylsilyloxide).

### SUMMARY OF THE INVENTION

In one aspect, the invention provides a dye-loaded nanoparticle comprising a phthahalocyanine (PC) dye, naphthalocyanine (NC) dye, or combination thereof solubilized within a micelle, polymersome, or liposome.

In another aspect, the invention provides a method for preparing a dye-loaded nanoparticle, the method comprising a) solubilizing a phthalocyanine (PC) dye, an naphthalocyanine (NC) dye, or combination thereof, within an amphiphilic polymer or a lipid to form dye-loaded nanoparticle micelles, polymersomes, or liposomes; and b) purifying the PC-loaded nanoparticulate micelles by dialysis, size exclusion chromatography, filtration, or any combination thereof.

In one aspect, the invention provides a method for visualization of a tumor without optical imaging equipment, the method comprising a) injecting a subject with dye-loaded nanoparticles; and b) detecting by direct visualization, camera, or endoscopy an accumulation of colored dye in a tumor and a demarcation of tumor margins.

In another aspect, the invention provides a method for visualization of a tumor with photoacoustic imaging equipment, the method comprising a) injecting a subject with dye-loaded nanoparticles; and b) detecting by photoacoustic imaging an accumulation of dye in a tumor and a demarcation of tumor margins.

In one aspect, the invention provides a method for detecting colorectal cancer, the method comprising a) injecting a subject with phthalocyanine (PC)-loaded nanoparticulate amphiphilic polymer or lipid micelles; and b) detecting by endoscopy an accumulation of blue dye in a tumor and a demarcation of tumor margins.

In another aspect, the invention provides a method for detecting ovarian or breast cancer, the method comprising a) injecting a subject with phthalocyanine (PC)-loaded nanoparticulate amphiphilic polymer or lipid micelles; and b) detecting by intraoperative photoacoustic imaging an accumulation of blue dye in a tumor and a demarcation of tumor margins.

In one aspect, the invention provides a phthalocyanine (PC)-loaded nanoparticulate polymeric or lipid micelle comprising a PC dye solubilized within the micelle as an oil-in-water emulsion.

In another aspect, the invention provides a method for preparing phthalocyanine (PC)-loaded nanoparticulate polymeric or lipid micelles, the method comprising: a) solubilizing a phthalocyanine (PC) dye with an amphiphilic polymer or a lipid to form PC-loaded nanoparticulate micelles; and b) purifying the PC-loaded nanoparticulate micelles by dialysis.

In one aspect, the invention provides a method for visualization of a tumor without optical imaging equipment, the method comprising: a) injecting a subject with phthalocyanine (PC)-loaded nanoparticulate amphiphilic polymer or lipid micelles; and b) detecting by endoscopy or intraoperative photoacoustic imaging an accumulation of blue dye in a tumor and a demarcation of tumor margins. The methods further comprise resecting the tumors and treating the resected organ (or an unresected organ in which a tumor has been) with photodynamic therapy (PDT) and/or photothermal therapy (PTT).

In another aspect, the invention provides a method for detecting colorectal cancer, the method comprising: a) injecting a subject with phthalocyanine (PC)-loaded nanoparticulate amphiphilic polymer or lipid micelles; and b) detecting by endoscopy an accumulation of blue dye in a tumor and a demarcation of tumor margins.

In one aspect, the invention provides a method for detecting ovarian or breast cancer, the method comprising: a) injecting a subject with phthalocyanine (PC)-loaded nanoparticulate amphiphilic polymer or lipid micelles; and b) detecting by intraoperative photoacoustic imaging an accumulation of blue dye in a tumor and a demarcation of tumor margins.

In another aspect, the invention provides a method for treating a tumor, the method comprises a) administering intravenously to a subject phthalocyanine (PC) dye-loaded or naphthalocyanine (NC) dye-loaded nanoparticulate amphiphilic polymer or lipid micelles; b) detecting by intraoperative photoacoustic imaging an accumulation of dye in a tumor and a demarcation of tumor margins; and/or c) irradiating the PC dye or the NC dye within the tumor with a laser to heat the tumor, thereby decreasing tumor cell viability and/or killing the tumor cells.

Other features and advantages will become apparent from the following detailed description, examples, and figures. It should be understood, however, that the detailed description and the specific examples while indicating preferred embodiments of the invention are given by way of illustration only, since various changes and modifications within the spirit and scope of the invention will become apparent to those skilled in the art from this detailed description.

### BRIEF DESCRIPTION OF THE FIGURES

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings.

FIGS. 1A-1H show the preparation, characterization and uses of the PC dye micelles according to the invention. FIG. 1A shows a scheme depicting the process of solubilizing hydrophobic PCs with PEG-PCL using oil-in-water (O/W) procedure, e.g., ZnPC-blue nanoparticles. FIG. 1B shows photographs of PCs loaded micelles with their chemical structure, hydrodynamic size, and PDI. FIG. 1C shows normalized signal-to-background ratio (SBR) measurements were made using the candidate tumors (e.g., photographs top of the bar chart) and its surrounding tissue as background after 24 h of PCs loaded micelles (10 mg/kg) and MB (10 mg/kg) intravenous (retro-orbital) injection (n=3). FIG. 1D shows ultrasound and photoacoustic imaging of tumor-bearing mice breast and FIG. 1E shows colon cancer. FIG. 1F shows temperature curves of ZnPC and NiPC loaded micelles (20 ug/mL) in water during exposure to 808 nm laser (2 W/cm²) over the period of 10 min. Infrared thermal images of water and NiPC micelle solution under laser irradiation. FIG. 1G shows dynamic light scattering profile of ZnPC-blue nanoparticles. FIG. 1H shows Absorbance spectra of ZnPC-blue nanoparticles in water.

FIGS. 2A-2D show hydrodynamic size of the ZnPC(TB) loaded micelles were monitored for 7 days in water and PBS at room temperature (FIGS. 3A-3B). (FIG. 3C) shows viability of 4T1 cells (black bar) and HT-29 cells (gray bar) after incubation with increasing concentrations of ZnPC(TB) loaded micelle for 24 h. FIG. 3D shows a bar chart shows the average zeta potential of the PCs loaded micelles in water.

FIG. 3A-3B show an illustration of a mouse injected with ZnPC(TB) nanoparticles and accumulation of blue dye in colon tumors (Left) and photographs of the excised colons (top) and small intestines (bottom) with inflammatory polyps pre and post-injection (24 h) of 20 mg/kg ZnPC(TB)-blue nanoparticles. FIG. 3B shows representative dissected mouse gastrointestinal tract. The photograph shows blue nanoparticles stained stomach sections, which are representative stomach lesions. Compared to the stomach from a control mouse, the submucosal region of the injected mouse (i.e., stomach) was observed as being significantly accumulated by the blue nanoparticles. Pre-injection versus post-injection SBR measurements is shown. The unpaired t-test was used for analyses. *P, **P, and ***P<0.001. Statistical significance was defined as P<0.05.

FIGS. 4A-4F show phthalocyanine dyes used in this study.

FIGS. 5A-5C show photographs of PCs and PCs loaded micelles (0.5 mM) in water and the retention of dyes according to the invention. Methylene blue (MB) is the only water-soluble blue dye that is used in this study. FIG. 5B shows normalized absorption spectra of the PCs loaded micelles and MB in water. FIG. 5C shows retention of dyes of varying hydrophobicity solubilized by PEG-PCL and then dialyzed against 20 mM cholate for 24 h.

FIGS. 6A-6B show photographs before (control) and 24 h after injection of 5 and 10 mg/kg ZnPC(TB) loaded micelles by ZnPC(TB) weight into a mouse bearing 4T1 flank xenograft tumor (n=3). (B) Pre-injection versus post-injection SBR measurements is shown. The unpaired t-test was used for analyses. Statistical significance was defined as P<0.05.

FIGS. 7A-7D show photographs of colons (top) and small intestines (bottom) excised from Apc^{+/Min-FCCC} mice with 10 mg/kg and 20 mg/kg injection of ZnPC(TB) blue nanoparticles. Pre-injection versus post-injection SBR measurements are shown. *P, and **P<0.001.

FIGS. 8A-8B show the concept of contrast-enhanced optical imaging of CRC. In contrast to WLE, very small premalignant lesion and flat polyps can be detected. Comparison of polyps detectability between conventional (WLE (FIG. 8A) and enhanced optical endoscopy (FIG. 8B).

FIGS. 9A-9B respectively show White light and photoacoustic imaging of a colon excised from an Apc^{+/min} mouse. Ex vivo photoacoustic imaging demonstrated (flat) lesions at the colon (yellow arrowhead).

FIGS. 10A-10B show methylene blue-aided chromaendoscopy. Tumor site as either a Flat lesion (FIG. 10A) or an Inflammatory polyp (FIG. 10B) denoted with red circle. The disadvantage of the technique is the often-inhomogeneous staining pattern and distinguish between lesion/polyp, and healthy tissue is difficult (i.e., tumor margin is not clear).

FIGS. 11A-11C show ZnPC-loaded micelles and visualized flank tumor margin pre-injection (FIG. 11B) and 24 h post-injection of ZnPC-loaded micelles (FIG. 11C).

FIGS. 12A-12B show examples of frozen sections of a flank tumor tissue (magnification 40×). Black arrows point to the presence of the blue color from the ZnPC(TB)-loaded micelles.

FIGS. 13A-13D show frozen tissues embedded in optimal cutting temperature compound (OCT) that were sectioned at an average cut depth of ˜0.5-1 mm using a hand microtome. FIG. 13A shows tumor tissues were embedded with OCT and then were cut using a hand and table microtome. It is possible to cut 0.5-1 mm thick slices reproducibly. (FIG. 13B) Flank, (FIG. 13C) colon and (FIG. 1dD) small intestine were live imaged using a microscope with a 10× objective. Scale Bars=220 μm.

FIGS. 14A-14C show photograph (scale bars=3 mm), color, bright-field, and fluorescence images of (FIG. 12A) flank, (FIG. 12B) colon, and (FIG. 12C) small intestine tissues. Microscope images were acquired with a 4× objective (scale bars=550 μm). The white dashed lines indicate the approximate tumor margin. Cancer tissues encircled by the dashed lines appear blue-green, and in the fluorescence imaging can be easily distinguished from normal tissue.

FIG. 15 shows an illustration of mouse injected with ZnPC nanoparticles and accumulation of blue dyes in colon tumors. Photographs of the excised colons (top) and small intestines (bottom) with inflammatory polyms after 24 hrs injection of 0, 10, and 20 mg/kg ZnPC-blue nanoparticles

FIGS. 16A-16D show a schematic representation of Pc—and Nc-loaded micelles (FIG. 16A), Pc—and Nc-loaded micelles in water (FIG. 16B), general structure of Ncs, Pcs (FIG. 16C), and PEG-PCL and absorbance Spectra of Nc—and Pc-loaded micelles in water (FIG. 16D).

FIGS. 17A-17C show a comparison of the heating ability of NCs, PCs and indocyanine green (ICG) dye, photoacoustic spectra of NCs and PCs, and photocytotoxicity of Pc—and Nc-loaded micelles. FIG. 17A shows solutions of various micelles in water (9.35 μM Pc/Nc), irradiated in multiple 10-minute increments, and cooled to room temperature before successive rounds of heating and cooling. FIG. 17B shows photoacoustic spectra of Nc—and Pc-loaded micelles in water. FIG. 17C shows photocytotoxicity of Pc—and Nc-loaded micelles incubated with ID8-Luciferace cells and irradiated for 5 minutes with 1 W using an 808 nm laser.

FIGS. 18A-18C show photographs of the micelles loaded with different Pc/Nc and their hydrodynamic size and PDI (FIG. 18A), temperature change curves of Pc/Nc-micelles (10 uM) in water during exposure to 808 nm laser (0.75W/cm2) over the period of 10 min (FIG. 18B). Complete photoacoustic spectra of Pc/Nc-micelles (FIG. 18C).

FIGS. 19A-19C show nanoparticle preparation. A schematic illustrates the formation of Pc/Nc-loaded micelles. Pc and/or Nc and the diblock copolymer PEG-PCL were first dissolved in organic solvent(s). Upon addition to water an oil-in-water emulsion was produced, whereby micelles were formed with the Pc and/or Nc encapsulated within the hydrophobic core of the polymeric micelles. (FIG. 19A). Representative photographs of Pc/Nc-loaded micelles, which were prepared using different Pc/Nc dyes (FIG. 19B); Transmission electron microscopy images of micelles loaded with CuNc Octa or MgPc (FIG. 19C).

FIG. 20 shows the UV absorbance spectra of six different micelles formulations, each loaded with a different Pc dye.

FIG. 21 shows the heating curves of the micelles according to embodiments of the invention. Pc and Nc-loaded micelles (9.4 uM dye concentration) and controls (empty micelles and water) were prepared and then irradiated at 808 nm (0.75 W/cm2) continuously for 10 min. Heating of each sample was measured as a function of time.

FIG. 22 shows the thermal stability of the micelles according to embodiments of the invention. Various Pc and Nc-loaded micelles (9.4 uM) were heated multiple times, using a laser (0.75 W/cm2), in 10-min increments. The samples were allowed to cool to room temperature between successive rounds.

FIG. 23 shows cell viability. An MTS cell proliferation assay of 4T1 cells after incubation with Nc or Pc-loaded micelles or standard media for 24 h. No significant toxicity was observed in the absence of laser irradiation; however, upon laser irradiation at 808 nm (0.75 W/cm2) for 5 min, each micelle formulation tested led to near complete cell killing, with the exception of ZnNc-loaded micelles, which exhibited very little cell killing.

FIG. 24 shows heating curves of the micelles according to embodiments of the invention in vivo. 4T1-tumor bearing mice had various Pc or Nc-loaded micelles administered intravenously (10 mg/kg). After 24 hours, the tumors were irradiated with a laser (808 nm, 0.75W) for 30 min. The change in tumor temperature was monitored via thermal imaging as a function of time.

FIGS. 25A-25B show photoacoustic (PA) spectra of the micelles according to embodiments of the invention. FIG. 25A shows PA spectra of various Nc-loaded micelle formulations. FIG. 25B shows PA spectra of various Pc-loaded micelle formulations.

FIG. 26 shows representative photoacoustic images of 4T1 flank tumors in mice 24 hours after intravenous administration of SiPC-, CuNC-, VaNC-, and Octa NC-loaded micelles. Control animals did not receive any contrast agents. Left, ultrasound image; right, photoacoustic (color) images.

FIGS. 27A-27B show PTT efficacy and safety using CuNc Octa micelles according to embodiments of the invention. Mice bearing 4T1 breast flank tumors were injected with saline, CuNc Octa-loaded micelles (NP, 10 mg/kg, no laser), or CuNc Octa (10, 5, 1 mg/kg, with laser) (n=5 per group). The subset of mice that received subcutaneous laser irradiation (0.7 W/cm2, 10 min) received this laser treatment 24 hrs following the injection of the micelles. The tumor size of all mice was monitored as a function of time (FIG. 27A). The body weight of all mice was also monitored as a function of time (FIG. 27A B).

### DETAILED DESCRIPTION OF THE INVENTION

The present subject matter may be understood more readily by reference to the following detailed description that forms a part of this disclosure. It is to be understood that this invention is not limited to the specific products, methods, conditions or parameters described and/or shown herein, and that the terminology used is for the purpose of describing particular aspect and embodiments by way of example only and is not intended to be limiting of the claimed invention.

Patients with inflammatory bowel disease (IBD) are at an increased risk of developing colorectal cancer (CRC). The current standard of care for surveillance of IBD has relied on white light endoscopy (WLE). However, there are still dysplastic lesions that are not visible to the naked eye using conventional WLE equipment. Dye-based chromoendoscopy (DBC) is an adjunct technique that has been investigated to enable endoscopists to visualize the colorectal mucosa better. However, inflammatory polyps exhibit similar dye uptake as the surrounding tissues, and current dyes do not significantly improve the recognition of tumor margins from the surrounding tissues.

To overcome these problems, nanoparticle-based delivery systems can be utilized. Polymeric micelles possess a supramolecular core-shell structure and represent a flexible nanoplatform for biomedical applications, holding hydrophobic compounds, e.g., drugs and dyes, inside the core of micelles. Characteristics such as controlled drug delivery, extended circulation time, reduced side effects, and biodegradability make these amphiphilic materials to be ideal nanocarriers for various agents. Loading with hydrophobic dyes makes the hydrophobic core of micelles more suitable for targeting, which increases their half-life and bioavailability. Dye-loaded micelles allow for the selective delivery of dyes into tumors while minimizing the uptake in normal tissue by the enhanced permeability and retention (EPR) effect.

Here, phthalocyanine (PC) blue-based nanoparticles were developed, resulting in an increased detection rate and improved delineation of tumor margins. These nanoparticles are expected to enable clinicians to distinguish between higher risk and lower risk patients and allow the colon to be more effectively cleared of dysplasia by reducing the likelihood of occult lesions that can grow and progress into a more threatening process.

In the study described herein, several hydrophobic phthalocyanine dyes (PCs) were tested and new contrast agents were developed for dye-based endoscopy that utilize PC dyes that can be delivered directly to the colorectal mucosa. Many PCs are at various phases of clinical trials, and miscellaneous strategies have been utilized to develop PCs with optimized photophysical properties, biocompatibility, dual therapeutic actions, and selective tumor targeting. In the study described herein, to enhance tumor margins detection, hydrophobic ZnPC blue dyes were loaded into biocompatible and biodegradable PEG-PCL micelle as a contrast agent that is visually detectable.

In one aspect, the invention provides a dye-loaded nanoparticle comprising a phthahalocyanine (PC) dye, naphthalocyanine (NC) dye, or combination thereof solubilized within a micelle, polymersome, or liposome. In an embodiment, the dye-loaded nanoparticle of claim 1, wherein the PC dye further includes octabutoxy, hexadecafluoro, tetrakis(4-cumylphenoxy), tetra-tert-butyl, tetraphenoxy, tetrakis(phenylthio) or octakis(octyloxy). In some embodiments of the PC dye, the PC dye is without or with a metal center and wherein the metal center comprises zinc, nickel, copper, iron, magnesium, aluminum, gallium, dilithium, titanyl, silicon, vanadyl, cobalt, tin, titanium, manganese, indium or lead. In certain embodiments, the NC dye further comprises alkyl groups. In an embodiment, the alkyl groups comprise an octabutoxy, bis(trihexylsilyloxide) or a tetra-tert-butyl group. In some embodiments, the NC dye is without or with a metal center and wherein the metal center includes zinc, nickel, copper, gallium, silicon, vanadyl, cobalt, tin, titanium, manganese, indium or lead. In some embodiments of the dye-loaded nanoparticle, the PC dye is Zinc phthalocyanine (ZnPC). In some embodiments, the NC dye is Zinc naphthalocyanine (ZnNC). In certain embodiments, the dye is a mixture of Zinc phthalocyanine and Zinc naphthalocyanine (ZnPC-NC). In some embodiments of the dye-loaded nanoparticle, the nanoparticle comprises an amphiphilic polymer or lipid or combination thereof. In particular embodiments of the dye-loaded nanoparticle, the amphiphilic polymer is a diblock copolymer, and icomprises poly(ethylene glycol)-poly(caprolactone) (PEG-PCL), poly(ethylene glycol)-poly(]actide-co-glycolide) (PEG-PLGA), poly(ethylene glycol)-poly(lactic acid) (PEG-PLA), poly(ethylene glycol)-poly(β-benzyl-L-aspartate) (PEG-PBLA), poly(ethylene glycol)-poly(amino acid. In an embodiment of the dye-loaded nanoparticle, the lipid is a Pluronic, 1,2dipalmitoyl-sn-glycero-3-phosphocholine (DPPC), L-αphosphatidylcholine hydrogenated (HSPC), distearoyl phosphatidyl ethanolamine-polyethylene glycol (DSPE-PEG), or L-α-phosphitidylcholine (EggPC). In a particular embodiment of the dye-loaded nanoparticle, the dye:amphiphile ratio (w/w) is in the range of 1:10 to 6:1. In some embodiments of the dye-loaded nanoparticle, the nanoparticle is a micelle and the dye ZnPC:PEG-PCL ratio (w/w) is 1:2. In certain embodiments of the dye-loaded nanoparticle, the nanoparticle is further functionalized with a targeting agent. In a particular embodiment, the targeting agent is an antibody, antibody fragments, affibody, DARPin, nanobody, protein, peptide, aptamer, or small molecule. In certain embodiments, the phthalocyanine dye (PC) comprises 29H,31H-Phthalocyanine, Dilithium phthalocyanine, Titanyl phthalocyanine, Zinc phthalocyanine, Nickel(II) phthalocyanine, Copper(II) phthalocyanine, Iron(II) phthalocyanine, Vanadyl phthalocyanine, Cobalt(II) phthalocyanine, Lead(II) phthalocyanine, Aluminum phthalocyanine chloride, Silicon phthalocyanine dichloride, Gallium(III)-phthalocyanine chloride, Titanium(IV) phthalocyanine dichloride, Iron(III) phthalocyanine chloride, Manganese(II) phthalocyanine, Magnesium phthalocyanine, Indium(III) phthalocyanine chloride, Lead(II) tetrakis(4-cumylphenoxy)phthalocyanine, Cobalt(II) 1,2,3,4,8,9,10,11,15,16,17,18,22,23,24,25-hexadecafluoro-29H,31H-phthalocyanine, Vanadyl 2,9,16,23-tetraphenoxy-29H,31H-phthalocyanine, Zinc 1,2,3,4,8,9,10,11,15,16,17,18,22,23,24,25-hexadecafluoro-29H,31H-phthalocyanine, Zinc 2,3,9,10,16,17,23,24-octakis(octyloxy)-29H,31H-phthalocyanine, Nickel(II) 1,4,8,11,15,18,22,25-octabutoxy-29H,31H-phthalocyanine, Copper(II) 1,4,8,11,15,18,22,25-octabutoxy-29H,31H-phthalocyanine, Aluminum 1,8,15,22-tetrakis(phenylthio)-29H,31H-phthalocyanine chloride, Copper(II) 2,3,9,10,16,17,23,24-octakis(octyloxy)-29H,31H-phthalocyanine, Zinc 2,9,16,23-tetra-tert-butyl-29H,31H-phthalocyanine, Copper(II) 1,2,3,4,8,9,10,11,15,16,17,18,22,23,24,25-hexadecafluoro-29H,31H-phthalocyanine, Zinc 1,4,8,11,15,18,22,25-octabutoxy-29H,31H-phthalocyanine, Copper(II) 2,9,16,23-tetra-tert-butyl-29H,31H-phthalocyanine, 1,4,8,11,15,18,22,25-Octabutoxy-29H,31H-phthalocyanine, 2,9,16,23-Tetra-tert-butyl-29H,31H-phthalocyanine, 2,3,9,10,16,17,23,24-Octakis(octyloxy)-29H,31H-phthalocyanine or combinations thereof. In various embodiments, the naphthalocyanine (NC) dye comprises 2,3-Naphthalocyanine, Vanadyl 2,3-naphthalocyanine, Copper(II) 2,3-naphthalocyanine, Cobalt(II) 2,3-naphthalocyanine, Tin(II) 2,3-naphthalocyanine, Silicon 2,3-naphthalocyanine dihydroxide, Silicon 2,3-naphthalocyanine bis(trihexylsilyloxide), Nickel(II) 5,9,14,18,23,27,32,36-octabutoxy-2,3-naphthalocyanine, Copper(II) 5,9,14,18,23,27,32,36-octabutoxy-2,3-naphthalocyanine, Vanadyl 2,11,20,29-tetra-tert-butyl-2,3-naphthalocyanine, Zinc 2,11,20,29-tetra-tert-butyl-2,3-naphthalocyanine, 5,9,14,18,23,27,32,36-Octabutoxy-2,3-naphthalocyanine, 2,11,20,29-Tetra-tert-butyl-2,3-naphthalocyanine or combinations thereof.

In another aspect, the invention provides a method for preparing a dye-loaded nanoparticle, the method comprising a) solubilizing a phthalocyanine (PC) dye, an naphthalocyanine (NC) dye, or combination thereof, within an amphiphilic polymer or a lipid to form dye-loaded nanparticle micelles, polymersomes, or liposomes; and b) purifying the PC-loaded nanoparticulate micelles by dialysis, size exclusion chromatography, filtration, or any combination thereof. In an embodiment of the method, the micelles are formed in an oil-in-water emulsion. In some embodiments, the dye, polymer, lipid, or combination thereof are dissolved in the oil phase. In certain embodiments, the PC dye further comprises alkyl groups or substituted alkyl groups. In an embodiment, the alkyl groups or substituted alkyl groups comprise octabutoxy, hexadecafluoro, tetrakis(4-cumylphenoxy), tetra-tert-butyl, tetraphenoxy, tetrakis(phenylthio) or octakis(octyloxy) groups. In an embodiment, the PC dye is without or with a metal center and wherein the metal center includes zinc, nickel, copper, iron, magnesium, aluminum, gallium, dilithium, titanyl, vanadyl, cobalt, tin, titanium, manganese, indium or lead. In some embodiments, the NC dye further comprises octabutoxy, bis(trihexylsilyloxide) or tetra-tert-butyl groups. In an embodiment of the provided methods, the NC dye is without or with a metal center and wherein the metal center includes zinc, nickel, copper, gallium, silicon, vanadyl, cobalt, tin, titanium, manganese, indium or lead. In some embodiments, the PC dye is Zinc phthalocyanine (ZnPC). In an embodiment, the NC dye is Zinc naphthalocyanine (ZnNC). In various embodiments, the dye is a mixture of Zinc phthalocyanine and Zinc naphthalocyanine (ZnPC-NC). In particular embodiments, the nanoparticle comprises an amphiphilic polymer or lipid or combination thereof. In some embodiments, the amphiphilic polymer is a diblock copolymer, and comprises poly(ethylene glycol)-poly(caprolactone) (PEG-PCL), poly(ethylene glycol)-poly(lactide-co-glycolide) (PEG-PLGA), poly(ethylene glycol)-poly(lactic acid) (PEG-PLA), poly(ethylene glycol)-poly(β-benzyl-L-aspartate) (PEG-PBLA), or poly(ethylene glycol)-poly(amino acid). In certain embodiments, the lipid is a Pluronic, 1,2-dipalmitoyl-sn-glycero-3-phosphocholine (DPPC), L-α-phosphatidylcholine hydrogenated (HSPC), distearol phosphatidyl ethanolamine-polyethylene glycol (DSPE-PEG), or L-α-phosphatidylcholine (EggPC). In various embodiments, the dye:amphiphile ratio (w/w) is in the range of 1:10 to 6:1. In certain embodiments, the nanoparticle is a micelle and the dye ZnPC:PEG-PCL ratio (w/w) is 1:2.

In one aspect, the invention provides a method for visualization of a tumor without optical imaging equipment, the method comprising a) injecting a subject with dye-loaded nanoparticles; and b) detecting by direct visualization, camera, or endoscopy an accumulation of colored dye in a tumor and a demarcation of tumor margins. In another aspect, the invention provides a method for visualization of a tumor with photoacoustic imaging equipment, the method comprising a) injecting a subject with dye-loaded nanoparticles; and b) detecting by photoacoustic imaging an accumulation of dye in a tumor and a demarcation of tumor margins. In various embodiments of the provided methods, the dye-loaded nanoparticles are injected at a dye dose in the range of 1 mg/kg to 50 mg/kg. In an embodiment, the dye is visualized or detected one day after injection of the dye-loaded nanoparticles. In some embodiments, the tumor is a nonpolypoid or polypoid colorectal adenoma. In certain embodiments, the methods further comprise resecting the tumor within a 1 cm radius of the tumor margins. In an embodiment, the methods further comprise treating the site of the resected tumor with photodynamic therapy (PDT) photothermal therapy (PTT), sonodynamic therapy or combination thereof. In some embodiments, the PC dye is Zinc 2,3,9,10,16,17,23,24-octakis(octyloxy)-29H,31H-phthalocyanine (ZnPC(octakis)), Nickel(II) 1,4,8,11,15,18,22,25-octabutoxy-29H,31H-phthalocyanine (NiPC), Copper(II) phthalocyanine (CuPC), Zinc phthalocyanine (ZnPC) or Zinc 2,9,16,23-tetra-tert-butyl-29H,31H-phthalocyanine (ZnPC(TB)). In various embodiments, the PC dye is Zinc phthalocyanine (ZnPC). In particular embodiments, the nanoparticle comprises an amphiphilic polymer or lipid or combination thereof, and the amphiphilic polymer is diblock copolymer (ethylene glycol)-co-poly(caprolactone) (PEG-PCL). In some embodiments, the PC dye Zn(TB):PEG-PCL ratio (w/w) is in the range of 1:4 to 1:1. In particular embodiments, the PC dye Zn(TB):PEG-PCL ratio (w/w) is 1:2.

In one aspect, the invention provides a method for detecting colorectal cancer, the method comprising a) injecting a subject with phthalocyanine (PC)-loaded nanoparticulate amphiphilic polymer or lipid micelles; and b) detecting by endoscopy an accumulation of blue dye in a tumor and a demarcation of tumor margins. In various embodiments of the provided methods, the dye-loaded nanoparticles are injected at a dye dose in the range of 1 mg/kg to 50 mg/kg. In an embodiment, the dye is visualized or detected one day after injection of the dye-loaded nanoparticles. In some embodiments, the tumor is a nonpolypoid or polypoid colorectal adenoma. In certain embodiments, the methods further comprise resecting the tumor within a 1 cm radius of the tumor margins. In an embodiment, the methods further comprise treating the site of the resected tumor with photodynamic therapy (PDT) photothermal therapy (PTT), sonodynamic therapy or combination thereof. In some embodiments, the PC dye is Zinc 2,3,9,10,16,17,23,24-octakis(octyloxy)-29H,31H-phthalocyanine (ZnPC(octakis)), Nickel(II) 1,4,8,11,15,18,22,25-octabutoxy-29H,31H-phthalocyanine (NiPC), Copper(II) phthalocyanine (CuPC), Zinc phthalocyanine (ZnPC) or Zinc 2,9,16,23-tetra-tert-butyl-29H,31H-phthalocyanine (ZnPC(TB)). In various embodiments, the PC dye is Zinc phthalocyanine (ZnPC). In particular embodiments, the nanoparticle comprises an amphiphilic polymer or lipid or combination thereof. In an embodiment, the amphiphilic polymer is diblock copolymer (ethylene glycol)-co-poly(caprolactone) (PEG-PCL). In some embodiments, the PC dye Zn(TB):PEG-PCL ratio (w/w) is in the range of 1:4 to 1:1. In particular embodiments, the PC dye Zn(TB):PEG-PCL ratio (w/w) is 1:2.

In one aspect, the invention provides a method for detecting ovarian or breast cancer, the method comprising a) injecting a subject with phthalocyanine (PC)-loaded nanoparticulate amphiphilic polymer or lipid micelles; and b) detecting by intraoperative photoacoustic imaging an accumulation of blue dye in a tumor and a demarcation of tumor margins. In various embodiments of the provided methods, the dye-loaded nanoparticles are injected at a dye dose in the range of 1 mg/kg to 50 mg/kg. In an embodiment, the dye is visualized or detected one day after injection of the dye-loaded nanoparticles. In some embodiments, the tumor is a lobular carcinoma, ductal carcinoma, ductal carcinoma in situ, Paget's disease of the breast, Inflammatory Breast Cancer or a phyllodes tumor. In certain embodiments, the methods further comprise resecting the tumor within a 1 cm radius of the tumor margins. In an embodiment, the methods further comprise treating the site of the resected tumor with photodynamic therapy (PDT) photothermal therapy (PTT), sonodynamic therapy or combination thereof. In some embodiments, the PC dye is Zinc 2,3,9,10,16,17,23,24-octakis(octyloxy)-29H,31H-phthalocyanine (ZnPC(octakis)), Nickel(II) 1,4,8,11,15,18,22,25-octabutoxy-29H,31H-phthalocyanine (NiPC), Copper(II) phthalocyanine (CuPC), Zinc phthalocyanine (ZnPC) or Zinc 2,9,16,23-tetra-tert-butyl-29H,31H-phthalocyanine (ZnPC(TB)). In various embodiments, the PC dye is Zinc phthalocyanine (ZnPC). In particular embodiments, the nanoparticle comprises an amphiphilic polymer or lipid or combination thereof. In an embodiment, the amphiphilic polymer is diblock copolymer (ethylene glycol)-co-poly(caprolactone) (PEG-PCL). In some embodiments, the PC dye Zn(TB):PEG-PCL ratio (w/w) is in the range of 1:4 to 1:1. In particular embodiments, the PC dye Zn(TB):PEG-PCL ratio (w/w) is 1:2.

In one aspect, the invention provides a phthalocyanine (PC)-loaded nanoparticulate polymeric or lipid micelle comprising a PC dye solubilized within the micelle as an oil-in-water emulsion. In an embodiment of the PC dye-loaded polymeric or lipid micelle, the PC dye is Zinc 2,3,9,10,16,17,23,24-octakis(octyloxy)-29H,31H-phthalocyanine (ZnPC(octakis)), Nickel(II) 1,4,8,11,15,18,22,25-octabutoxy-29H,31H-phthalocyanine (NiPC), Copper(II) phthalocyanine (CuPC), Zinc phthalocyanine (ZnPC) or Zinc 2,9,16,23-tetra-tert-butyl-29H,31H-phthalocyanine (ZnPC(TB)). In certain embodiments, the PC dye is Zinc phthalocyanine (ZnPC). In some embodiments of the PC dye-loaded nanoparticulate polymeric or lipid micelle, the polymeric micelle comprises an amphiphilic polymer. In an embodiment of the PC dye-loaded nanoparticulate polymeric or lipid micelle, the amphiphilic polymer is diblock copolymer (ethylene glycol)-co-poly(caprolactone) (PEG-PCL). In certain embodiments of the PC dye-loaded nanoparticulate polymeric or lipid micelle, the PC dye Zn(TB):PEG-PCL ratio (w/w) is in the range of 1:4 to 1:1. In a particular embodiment of the PC dye-loaded nanoparticulate polymeric or lipid micelle, the PC dye Zn(TB):PEG-PCL ratio (w/w) is 1:2.

In another aspect, the invention provides a method for preparing phthalocyanine (PC)-loaded nanoparticulate polymeric or lipid micelles, the method comprising a) solubilizing a phthalocyanine (PC) dye with an amphiphilic polymer or a lipid to form PC-loaded nanoparticulate micelles; and b) purifying the PC-loaded nanoparticulate micelles by dialysis. In some embodiments of the method for preparing phthalocyanine (PC)-loaded nanoparticulate polymeric or lipid micelles, the PC dye is Zinc 2,3,9,10,16,17,23,24-octakis(octyloxy)-29H,31H-phthalocyanine (ZnPC(octakis)), Nickel(II) 1,4,8,11,15,18,22,25-octabutoxy-29H,31H-phthalocyanine (NiPC), Copper(II) phthalocyanine (CuPC), Zinc phthalocyanine (ZnPC) or Zinc 2,9,16,23-tetra-tert-butyl-29H,31H-phthalocyanine (ZnPC(TB)). In a particular embodiment of the methods provided herein, the PC dye is Zinc phthalocyanine (ZnPC). In some embodiments of the methods, the amphiphilic polymer is diblock copolymer (ethylene glycol)-co-poly(caprolactone) (PEG-PCL). In certain embodiments of said methods, the PC dye Zn(TB):PEG-PCL ratio (w/w) is in the range of 1:4 to 1:1. In some embodiments of the methods, the PC dye Zn(TB):PEG-PCL ratio (w/w) is 1:2.

In one aspect, the invention provides a method for visualization of a tumor without optical imaging equipment, the method comprising a) injecting a subject with phthalocyanine (PC)-loaded nanoparticulate amphiphilic polymer or lipid micelles; and b) detecting by endoscopy an accumulation of blue dye in a tumor and a demarcation of tumor margins. In certain embodiments of the method for visualization of a tumor without optical imaging equipment, the phthalocyanine (PC)-loaded nanoparticulate polymer or lipid micelles are injected at a dose of 20 mg/kg. In an embodiment, the endoscopy is performed 24 hours after injection of the PC-loaded nanoparticulate polymeric or lipid micelles. In some embodiments of the methods, the tumor is a nonpolypoid or polypoid colorectal adenoma. In an embodiment of the methods for visualization of a tumor without optical imaging equipment, the method further comprises resecting the tumor within a 1 cm radius of the tumor margins. In certain embodiments of said methods, the PC dye is Zinc 2,3,9,10,16,17,23,24-octakis(octyloxy)-29H,31H-phthalocyanine (ZnPC(octakis)), Nickel(II) 1,4,8,11,15,18,22,25-octabutoxy-29H,31H-phthalocyanine (NiPC), Copper(II) phthalocyanine (CuPC), Zinc phthalocyanine (ZnPC) or Zinc 2,9,16,23-tetra-tert-butyl-29H,31H-phthalocyanine (ZnPC(TB)). In some embodiments of the methods, the PC dye is Zinc phthalocyanine (ZnPC). In a particular embodiment of the methods provided herein, the amphiphilic polymer is diblock copolymer (ethylene glycol)-co-poly(caprolactone) (PEG-PCL). In various embodiments, the PC dye Zn(TB):PEG-PCL ratio (w/w) is in the range of 1:4 to 1:1. In certain embodiments of said methods, the PC dye Zn(TB):PEG-PCL ratio (w/w) is 1:2.

In another aspect, the invention provides a method for detecting colorectal cancer, the method comprising a) injecting a subject with phthalocyanine (PC)-loaded nanoparticulate amphiphilic polymer or lipid micelles; and b) detecting by endoscopy an accumulation of blue dye in a tumor and a demarcation of tumor margins. In an embodiment of method for detecting colorectal cancer, the phthalocyanine (PC)-loaded nanoparticulate polymer or lipid micelles are injected at a dose of 20 mg/kg. In some embodiments of the methods, the endoscopy is performed 24 hours after injection of the PC-loaded nanoparticulate polymeric or lipid micelles. In various embodiments, the tumor is a nonpolypoid or polypoid colorectal adenoma. In some embodiments of the methods provided herein, the method further comprises resecting the tumor within a 1 cm radius of the tumor margins. In certain embodiments of said methods, the PC dye is Zinc 2,3,9,10,16,17,23,24-octakis(octyloxy)-29H,31H-phthalocyanine (ZnPC(octakis)), Nickel(II) 1,4,8,11,15,18,22,25-octabutoxy-29H,31H-phthalocyanine (NiPC), Copper(II) phthalocyanine (CuPC), Zinc phthalocyanine (ZnPC) or Zinc 2,9,16,23-tetra-tert-butyl-29H,31H-phthalocyanine (ZnPC(TB)). In a particular embodiment of the methods provided herein, the PC dye is Zinc phthalocyanine (ZnPC). In various embodiments of the methods, the amphiphilic polymer is diblock copolymer (ethylene glycol)-co-poly(caprolactone) (PEG-PCL). In some embodiments of the methods provided herein, the PC dye Zn(TB):PEG-PCL ratio (w/w) is in the range of 1:4 to 1:1. In a particular embodiment of the methods, the PC dye Zn(TB):PEG-PCL ratio (w/w) is 1:2.

In one aspect, the invention provides a method for detecting ovarian or breast cancer, the method comprising a) injecting a subject with phthalocyanine (PC)-loaded nanoparticulate amphiphilic polymer or lipid micelles; and b) detecting by intraoperative photoacoustic imaging an accumulation of blue dye in a tumor and a demarcation of tumor margins. In an embodiment of method for detecting ovarian or breast cancer, the phthalocyanine (PC)-loaded nanoparticulate polymer or lipid micelles are injected at a dose of 20 mg/kg. In some embodiments of the methods provided herein, the endoscopy is performed 24 hours after injection of the PC-loaded nanoparticulate polymeric or lipid micelles. In certain embodiments of said methods, the method further comprises resecting the tumor within a 1 cm radius of the tumor margins. In a particular embodiment of the methods provided herein, the method further comprises treating the resected ovarian or breast with photodynamic therapy (PDT) and photothermal therapy (PTT). In some embodiments of the methods, the PC dye is Zinc 2,3,9,10,16,17,23,24-octakis(octyloxy)-29H,31H-phthalocyanine (ZnPC(octakis)), Nickel(II) 1,4,8,11,15,18,22,25-octabutoxy-29H,31H-phthalocyanine (NiPC), Copper(II) phthalocyanine (CuPC), Zinc phthalocyanine (ZnPC) or Zinc 2,9,16,23-tetra-tert-butyl-29H,31H-phthalocyanine (ZnPC(TB)). In a particular embodiment of the methods, the PC dye is Zinc phthalocyanine (ZnPC). In certain embodiments of said methods, the amphiphilic polymer is diblock copolymer (ethylene glycol)-co-poly(caprolactone) (PEG-PCL). In various embodiments of the methods, the PC dye Zn(TB):PEG-PCL ratio (w/w) is in the range of 1:4 to 1:1. In some embodiments of the methods In some embodiments of the methods, the PC dye Zn(TB):PEG-PCL ratio (w/w) is 1:2.

In another aspect, the invention provides a method for treating a tumor, the method comprises a) administering intravenously to a subject phthalocyanine (PC) dye-loaded or naphthalocyanine (NC) dye-loaded nanoparticulate amphiphilic polymer or lipid micelles; b) detecting by intraoperative photoacoustic imaging an accumulation of dye in a tumor and a demarcation of tumor margins; and/or c) irradiating the PC dye or the NC dye within the tumor with a laser to heat the tumor, thereby decreasing tumor cell viability and/or killing the tumor cells. In an embodiment, the PC dye is ZnPc Octa, CuPc Octa, NiPc Octa, OctaPc, VaPc Tetra, PbPc Tetra, GaPc, TBPc, SiPc TB-OH, ZnPc Octakis, Octakis Pc, ZnPc, VaPc, AlPc Tetra, MnPc, MgPc, DiLiPc, ZnPc TB, FePc, AlPc, SiPc-OH, SiPc-Cl, NiPc, InPc, TiOPc, CoPc or CuPc. In some embodiments, the PC dye is MgPC, VaPC, OctaPC or A1PC. In certain embodiments, the NC dye is OctaNc, ZnNc, CuNc Octa, VaNc TB, Nc CoNc, NiNc, SiNc-Cl or CuNc. In various embodiments, the NC dye is CuNC, NiNC, VaNC TB, and OctaNC. In an embodiment of the herein provided method for treating a tumor, the detecting by intraoperative photoacoustic imaging localize to the tumor and assess the entire margin and the irradiating are in real-time. In another embodiment of said methods, the tumor is heated to 39° C. to 70° C. within 10 minutes. In some embodiments, the tumor is an ovarian tumor or a breast cancer tumor. In certain embodiments, the tumor is a nonpolypoid or polypoid colorectal adenoma. In a particular embodiment, the tumor is a colorectal cancer. In some embodiments, the PC or NC dye is visualized or detected one day after intravenous administration of the dye-loaded nanoparticulate amphiphilic polymer or lipid micelles. In various embodiments, dye-loaded nanoparticulate comprises an amphiphilic polymer or lipid or combination thereof. In some embodiments, the amphiphilic polymer is a diblock copolymer, and includes poly(ethylene glycol)-poly(caprolactone) (PEG-PCL), poly(ethylene glycol)-poly(lactide-co-glycolide) (PEG-PLGA), poly(ethylene glycol)-poly(lactic acid) (PEG-PLA), poly(ethylene glycol)-poly(β-benzyl-L-aspartate) (PEG-PBLA), or poly(ethylene glycol)-poly(amino acid). In an embodiment, the lipid is a Pluronic, 1,2-dipalmitoyl-sn-glycero-3-phosphocholine (DPPC), L-α-phosphatidylcholine hydrogenated (HSPC), distearoyl phosphatidyl ethanolamine-polyethylene glycol (DPSE-PEG), or L-α-phosphatidylcholine (EGGPC). In particular embodiments, the wavelength of the laser is near the peak absorbance of the PC dye or the NC dye. In certain embodiments, the wavelength of the laser is 808 nm.

The following examples are presented in order to more fully illustrate the preferred embodiments of the invention. They should in no way be construed, however, as limiting the broad scope of the invention.

### Example 1

### Phthalocyanine-Blue Nanoparticles

### Methods and Materials

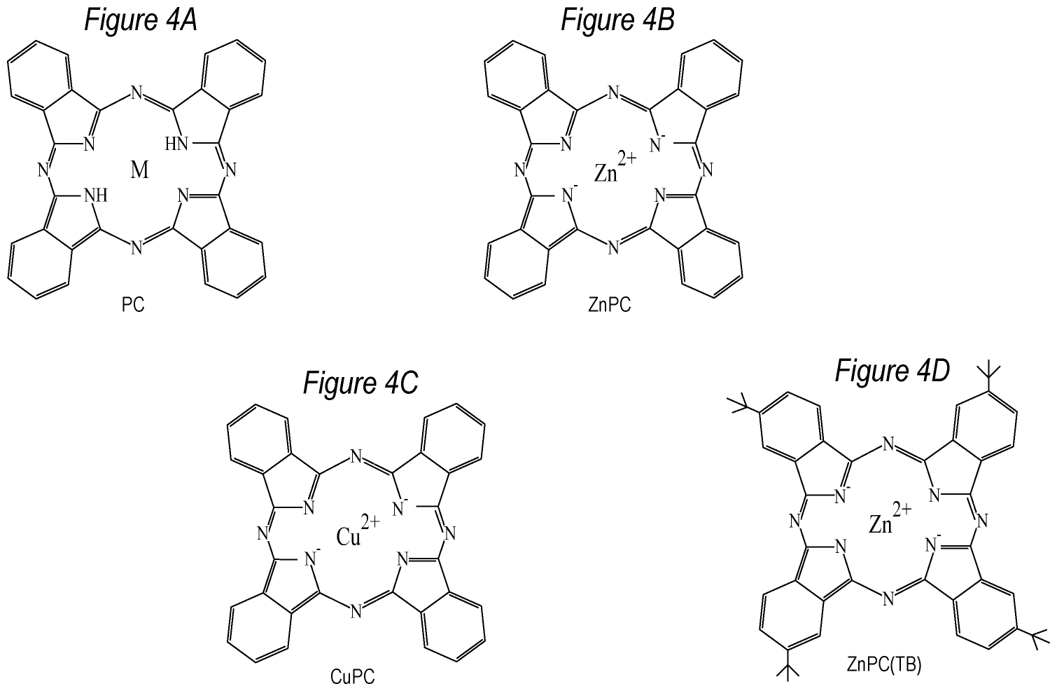

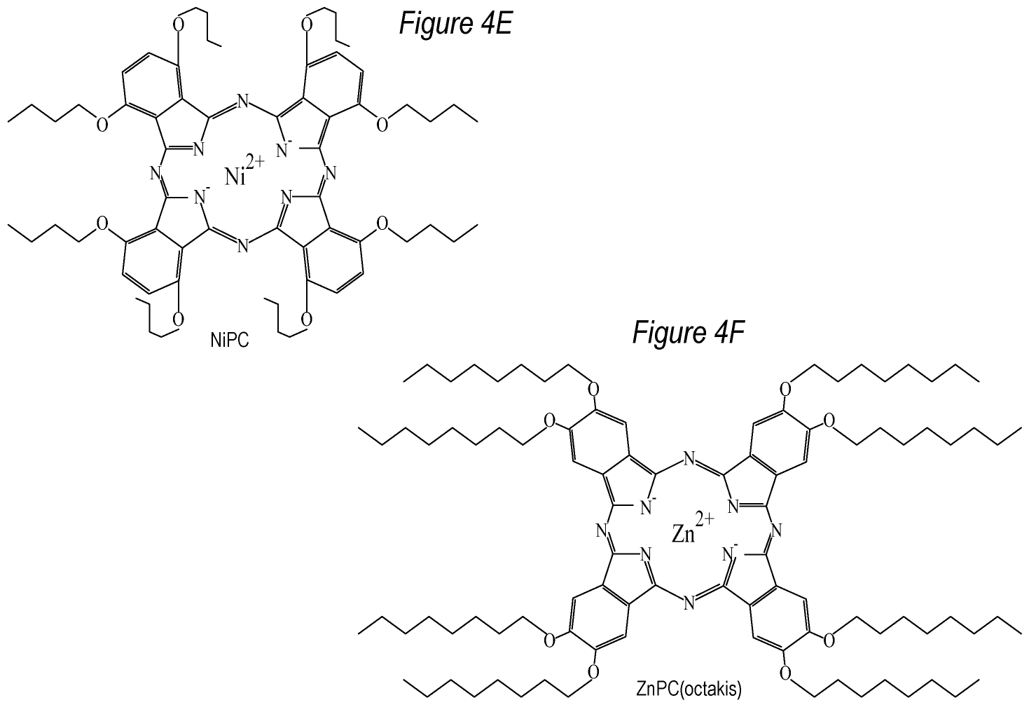

Methylene blue (MB), Zinc 2,3,9,10,16,17,23,24-octakis(octyloxy)-29H,31H-phthalocyanine (ZnPC(octakis)), Nickel(II) 1,4,8,11,15,18,22,25-octabutoxy-29H,31H-phthalocyanine (NiPC(octa)), Copper(II) phthalocyanine (CuPC), Zinc phthalocyanine (ZnPC), Zinc 2,9,16,23-tetra-tert-butyl-29H,31H-phthalocyanine (ZnPC(TB)) were purchased from Sigma, and 29H,31H-Phthalocyanine (PC) was obtained from Santa Cruz Biotechnology. Poly (ethylene glycol)-co-poly (caprolactone) (PEG-PCL) was obtained from Polymer Source Inc. Phthalocyanine dyes PC, ZnPC, CuPC, ZnPC(TB), NiPC, and ZnPC(octakis) used in this study are shown in FIGS. 4A-4F.

### Preparation of PCs nanoparticles

A mixture (200 μL) containing PCs dyes (2 mg into dimethyl sulfoxide (DMSO)) and amphiphilic diblock copolymer poly(ethylene glycol)-poly(ε-caprolactone) (PEG-PCL) (4 mg in toluene) was quickly added into a glass vial containing 4 mL of deionized water under sonication at room temperature until a homogeneous solution was observed. The toluene was evaporated overnight. Dialysis was performed with dialysis tubing (3500 MWCO) into 4L of deionized water to remove DMSO.

### Characterization of PCs Nanoparticles

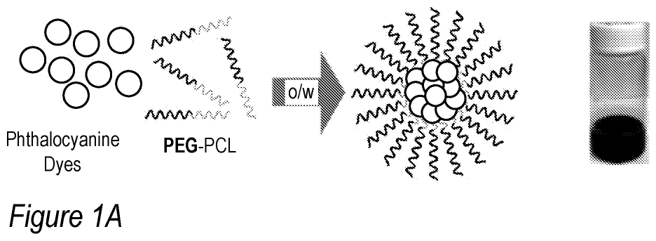

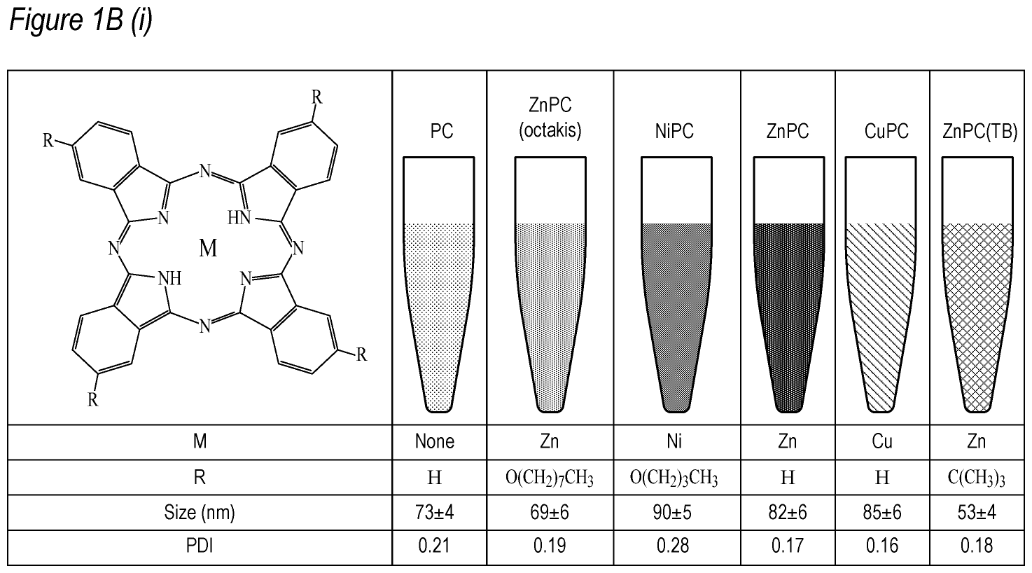

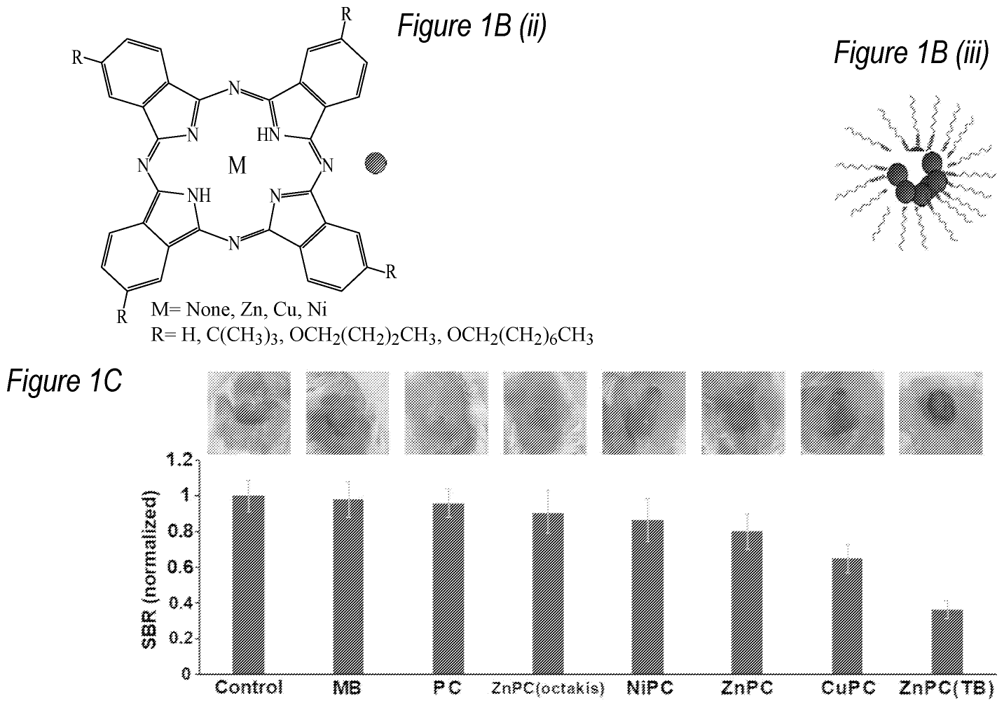

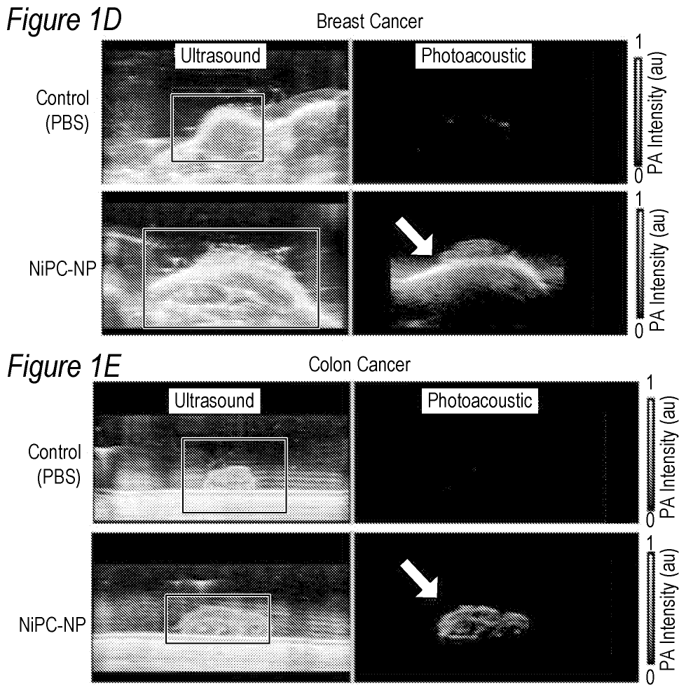

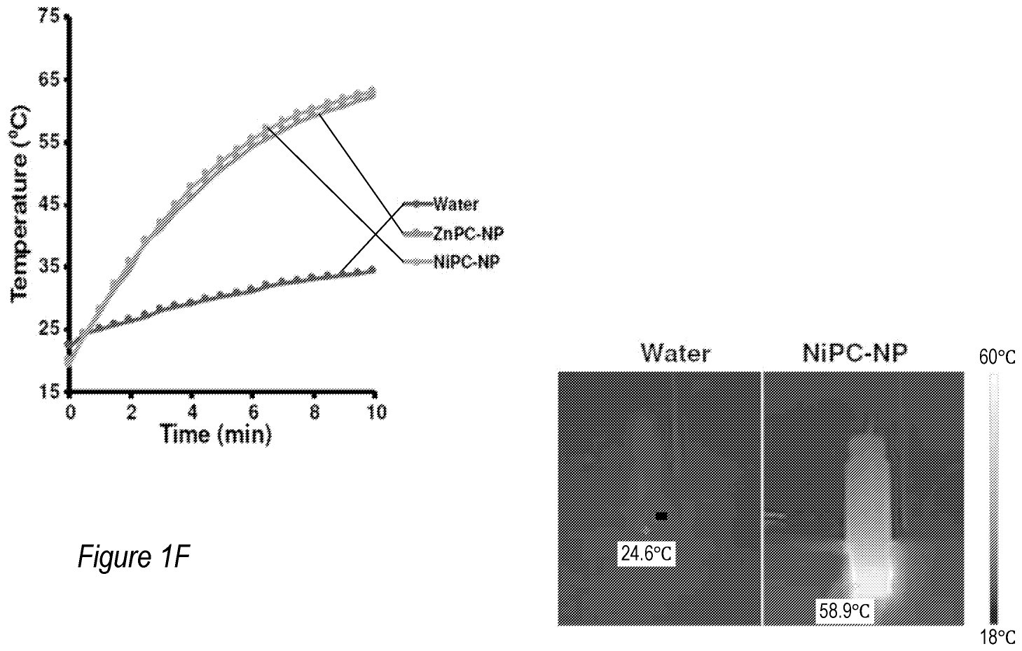

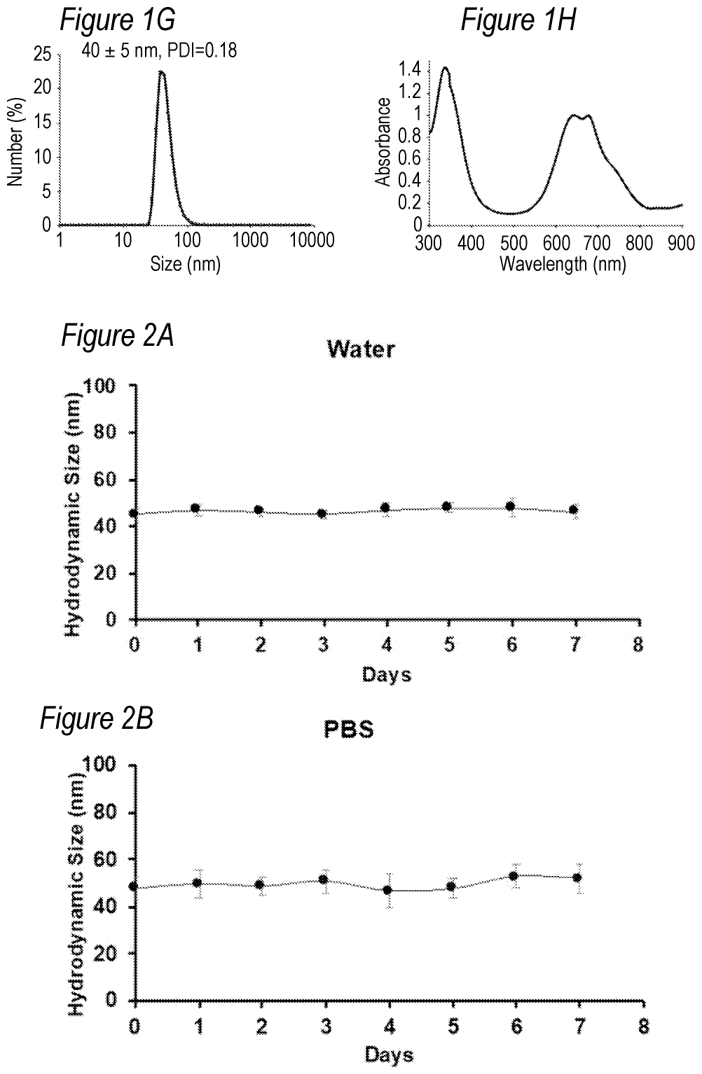

Various hydrophobic derivatives of the ‘blue’ dye Zinc Phthalocyanine (ZnPC) (e.g., with and without alkyl groups) were loaded into polymeric micelles, composed of the amphiphilic diblock copolymer poly (ethylene glycol)-co-poly(caprolactone) (PEG-PCL), via oil-in-water emulsions. The ZnPC-blue nanoparticles were characterized by UV-absorption spectroscopy and dynamic light scattering (FIGS. 1G-1H). The stability of the ZnPC nanoparticles was assessed in PBS and sodium cholate buffer by monitoring leakage of ZnPC dyes and the hydrodynamic diameter as a function of time. The cytotoxicity of ZnPC nanoparticles to 4T1 and HT-29 cells was then examined via an MTS cell proliferation assay. The contrast enhancing capabilities of ZnPC-blue nanoparticles were tested in a murine flank tumor model, using 4T1 cells and adenomatous polyposis coli (Apc)^{+/Min} mice that spontaneously develop multiple colorectal adenomas. FIG. 1G shows dynamic light scattering profile of ZnPC-blue nanoparticles. FIG. 1H shows absorbance spectra of ZnPC-blue nanoparticles in water.

The diameter and size distributions of the PCs nanoparticles were measured with dynamic light scattering (DLS, Malvern, Zetasizer, Nano-ZS). The encapsulation efficiency and payload of PC dyes were determined using a UV-Vis spectrophotometer (Varian, 100 Bio). See Table 1.

TABLE 1
Physical-chemical properties of ZnPC(TB) loaded micelles.

| ZnPC(TB):PEG-PCL Ratio | | | | | | |
|---|---|---|---|---|---|---|
| (w/w) | 1:4 | 1:2 | 1:1 | 2:1 | 4:1 | 20:1 |
| Encapsulation Efficiency (%) | 96.0 | 97.2 | 97.3 | 68.7 | 63.2 | 25.6 |
| Payload (%) | 19.2 | 32.4 | 48.7 | 45.8 | 50.6 | 24.4 |
| Hydrodynamic Diameter (nm) | 37 | 48 | 59 | 107 | 110 | 119 |
| Polydispersity Index | 0.215 | 0.175 | 0.195 | 0.234 | 0.180 | 0.265 |

PCs nanoparticles retention studies

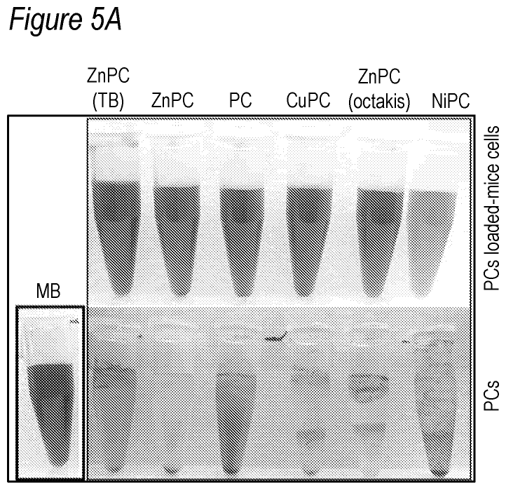

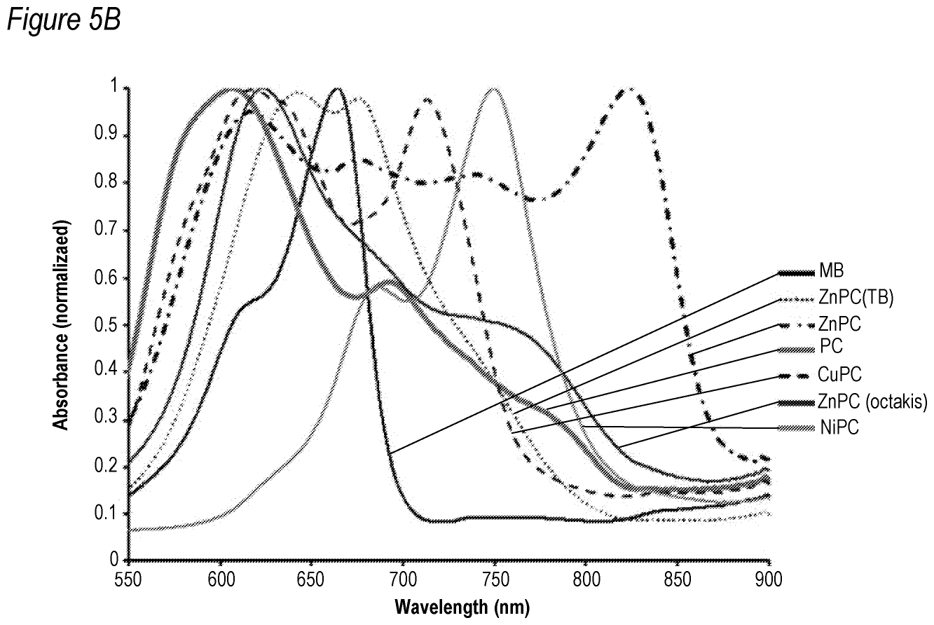

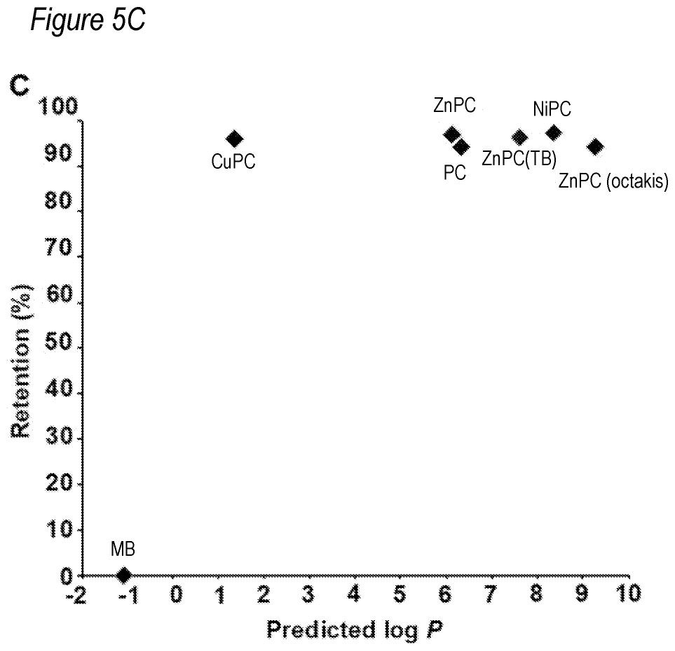

100 μL of PCs nanoparticles was diluted in 3 mL of 20 mM sodium cholate solution. Dialysis was performed with dialysis tubing (12,000-14,000 MWCO, Fisher) into 500 mL of 20 mM sodium cholate buffer at room temperature. The buffer was changed after 4 hours. The absorbance of the solutions was recorded before and after (24 hours) dialysis to determine dye retention percentage. FIG. 5A shows photographs of PCs and PCs loaded micelles (0.5 mM) in water. Methylene blue (MB) is the only water-soluble blue dye that is used in this study. FIG. 5B shows normalized absorption spectra of the PCs loaded micelles and MB in water. FIG. 5C shows retention of dyes of varying hydrophobicity solubilized by PEG-PCL and then dialyzed against 20 mM cholate for 24 h.

### MTS Assay

4T1 (triple-negative breast cancer) and HT-29 (colon cancer) cells (1×10⁴ cells per well) were seeded in 96-well plates and incubated overnight to allow the cells to attach to the surface of the wells. The cells were then mixed with increasing concentrations of ZnPC(TB) blue nanoparticles for 24 h, and the cell viabilities were determined using an MTS assay (Abcam Inc.) according to the supplier's instructions. Briefly, after 24 h of incubation with ZnPC(TB) blue nanoparticles, 10 ul of MTS reagent was added. After 2 h, absorbance was read at 490-nm on a Tecan microplate reader.

PCs nanoparticles were synthesized using amphiphilic diblock copolymer (PEG-PCL) (FIG. 1A, phthalocyanine dyes used in this study are shown in FIGS. 4A-4F, see methods in Example 1). Due to the hydrophobic nature of PCs, they tend to aggregate in the aqueous medium (intermolecular interaction (π-π stacking)). However, the micelles-based delivery system maintains the stability and activity of hydrophobic PCs (i.e., PCs-loaded micelles) in aqueous environments (FIG. 1B(i), FIGS. 5A-5C, photograph of PCs and PCs-loaded micelles in water). The size of nanoparticles was determined with dynamic light scattering (DLS). The size of nanoparticles was from 50 to 100 nm with the polydispersity index (PDI) around 0.2 (FIG. 1B(i)). UV-visible spectroscopy (FIG. 5B) provided evidence of solubility of PCs-loaded micelles in water. The release of the PCs from PCs loaded micelles was assessed in sodium cholate buffer by monitoring leakage of PCs dyes as a function of time. The solutions were dialyzed against the sodium cholate, as described in Zhang, Y.; et al. Nat. Nanotechnol. 2014, 9 (8), 631-638, which is incorporated by reference herein in its entirety. As shown in FIG. 5C, PCs that were very hydrophobic based on the octanol-water partition coefficient (log P values, estimated with the ALOGPS algorithm, as described in Tetko, I. V.; Tanchuk, V. Y. J. Chem. Inf. Comput. Sci. 2002, 42 (5), 1136-1145, which is incorporated by reference herein in its entirety) demonstrated high retention after dialysis (>95% in 24 h). Unlike nanoparticles, methylene blue (MB) was released entirely from the dialysis tubing.

### Results and Discussion

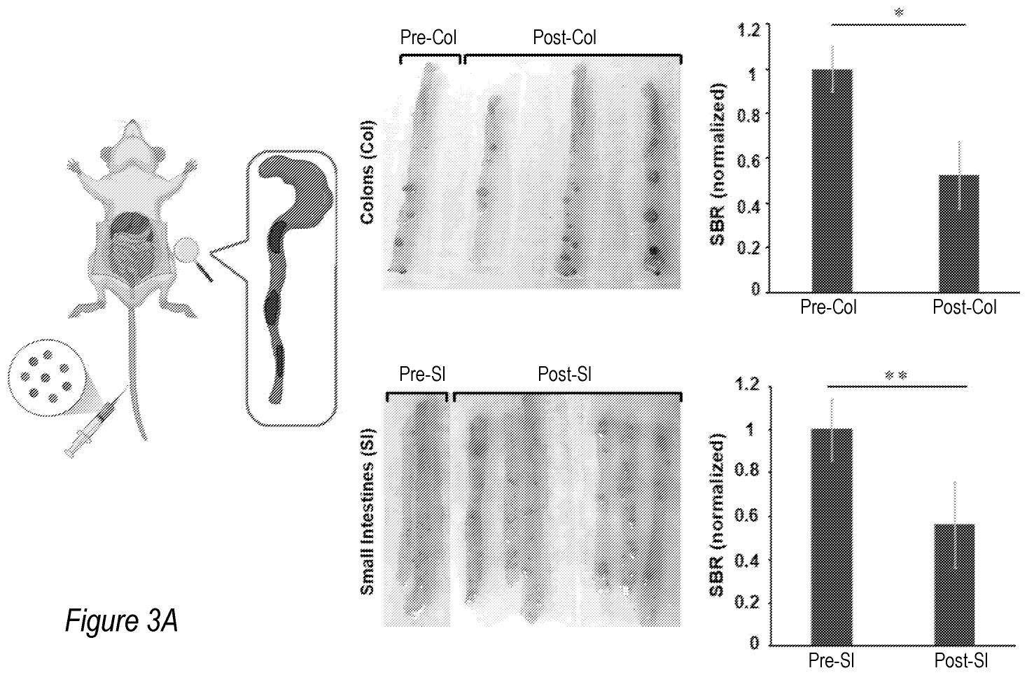

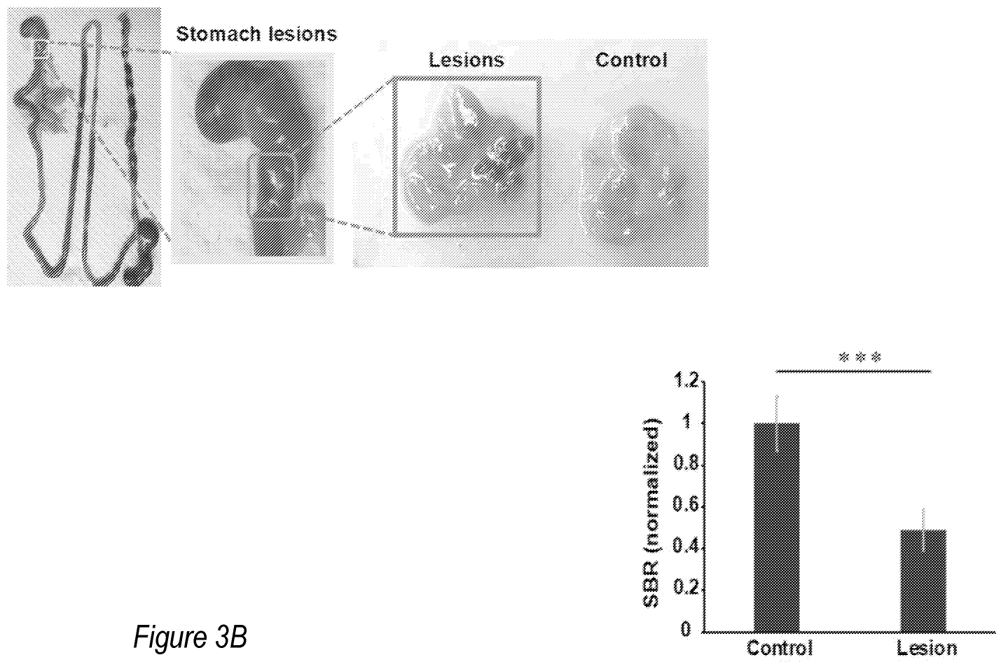

ZnPC-blue nanoparticles demonstrated size stability in water and PBS with high loading capacity (>90%), high retention after dialysis (>90% in 24 hrs in sodium cholate buffer) and narrow-size distribution (PDI<0.2). No toxicity was evident upon incubation with 4T1 and HT-29 cells up to a concentration of 100 μg/mL. Twenty-four hours post injection of 20 mg/kg ZnPC-blue nanoparticles (based on ZnPC dye weight) into (Apc)^{+/Min} mice revealed clear demarcation of tumor margins in excised colon and small intestine tissues, with efficient blue dye accumulation in polyps (See Example 2, FIGS. 3A-3B). Briefly, FIGS. 3A-3B. (Left) show an illustration of a mouse injected with ZnPC nanoparticles and accumulation of blue dye in colon tumors. Photographs of the excised colons (top) and small intestines (bottom) with inflammatory polyps after 24 hrs injection of 0, 10, and 20 mg/kg ZnPC-blue nanoparticles.

### Example 2

### Phthalocyanine-Blue Nanoparticle-Assisted White Light Endoscopy for Improved Detection of Colorectal Cancer

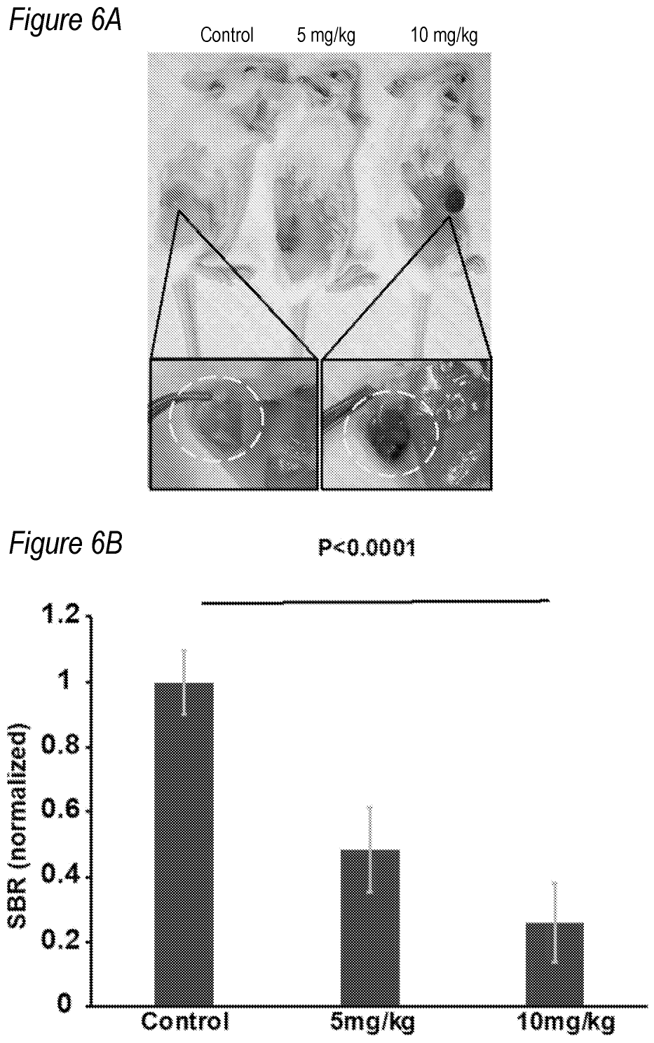

In preliminary studies in mice bearing 4T1 triple-negative breast flank xenograft tumors, it was found that ZnPC(TB)-loaded micelles (ZnPC(TB) blue nanoparticles) could be delivered to the tumor site and significantly improve the ability to accurately identify tumor margins rather than other PCs loaded micelles (FIG. 1C, FIG. 6A). Photographs were quantified using ImageJ. Normalized signal-to-background (SBR) measurements were made using the tumor (i.e., the intensity of the blue color) and its surrounding tissue as background after 24 h of PCs loaded micelles (10 mg/kg) and MB (10 mg/kg) intravenous (retro-orbital) injection (n=3). For a comparison, methylene blue (MB), clinically approved water-soluble blue dye, was also injected, and no accumulation was observed through the tumor sites because of the rapid clearance from the systemic circulation (FIG. 1C).

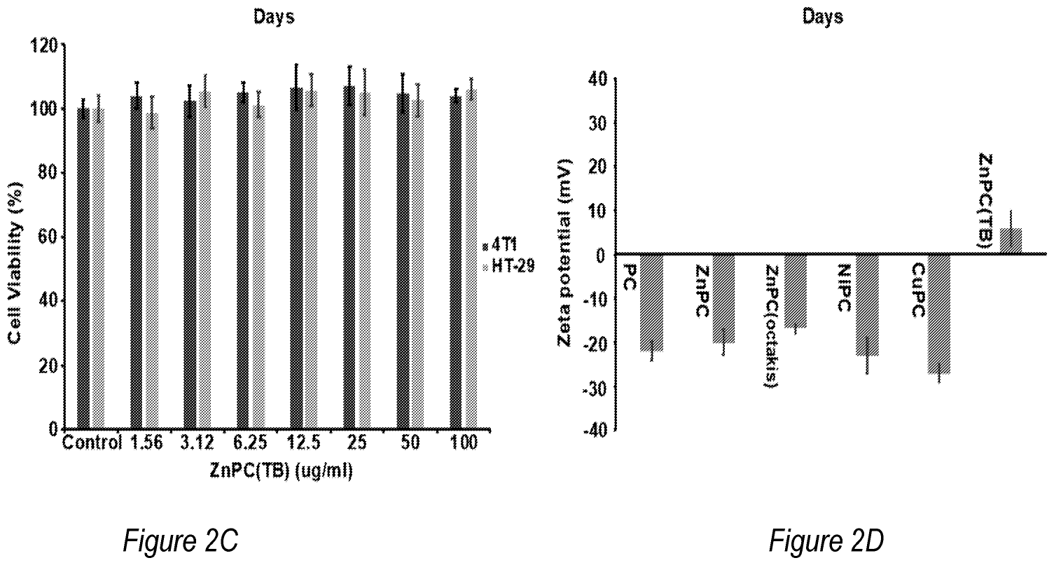

The post-injection SBR for ZnPC(TB) loaded micelles was significantly lower at 0.28±0.05 than the pre-injection SBR of 1.0±0.09 with 10 mg/kg after 24 h injection (FIG. 6B). These results indicated that ZnPC(TB) loaded micelle is a suitable candidate for tumor visualization. The preparation of ZnPC(TB) loaded micelles was highly reproducible and easily scalable. The effect of varying the amount of ZnPC(TB) dye, with a fixed amount of PEG-PCL, on the physical-chemical properties of the ZnPC(TB) loaded micelles was evaluated. It was found that the encapsulation efficiency is >95% for ZnPC(TB) when the Zn(TB):PEG-PCL ratio (w/w) is in the range of 1:4 to 1:1 (Table 1). The ZnPC(TB) payload was approximately 48% of the total weight (ZnPC(TB)+PEG-PCL) at 1:1 ratio. Increasing the ZnPC(TB):PEG-PCL ratio to 2:1 led to a reduction in encapsulation efficiency to 68%, but the payload increased to 50% of the total weight. In addition, the hydrodynamic diameter increased as the ZnPC(TB):PEG-PCL ratio increased. ZnPC(TB) loaded micelles formed using a ZnPC(TB):PEG-PCL ratio of 1:2 were used for all subsequent studies. ZnPC(TB) loaded micelle demonstrated size stability in water and PBS without aggregation or precipitation as indicated by no significant changes in hydrodynamic diameter for at least 7 days (FIGS. 2A and 2B).

To ensure that ZnPC(TB) loaded micelles can be safely injected into mice, the cytotoxicity of the ZnPC(TB) nanoparticles was examined in an MTS cell proliferation assay. Increasing concentration of ZnPC(TB) loaded micelles were incubated with cells for 24 h. It was found that the ZnPC(TB) loaded micelles exhibited no obvious cytotoxicity to 4T1 murine breast cancer cells or colon cancer (HT-29) cells (FIG. 2C) up to a concentration of 100 μg/mL.

The zeta potential for PCs loaded micelles was negative, except ZnPC(TB) loaded micelle, slightly positive (FIG. 2D). It has been reported that positive surface charge to be a significant factor in the increasing nanoparticle endocytosis. A positive surface charge facilitates target cell contact due to electrostatic interaction towards negatively charged proteoglycans located within the plasma membrane. In addition, negative charges on the endothelial cell membranes of the tumor vasculature might have potentiated the electrostatic interaction between positively charged nanoparticles and tumors. Thus, positive charges of nanoparticles at the tumor site and more negative charges of tumor cells/vasculature—appear to be responsible for the tumor-specific accumulation of ZnPC(TB) loaded micelle.

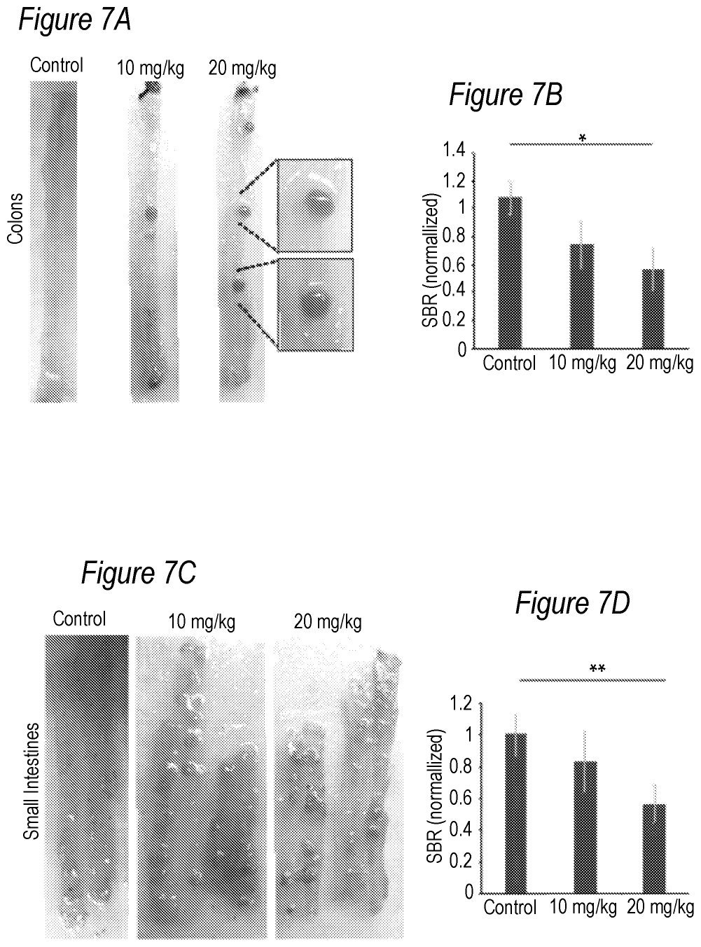

To assess the feasibility of using newly developed ZnPC(TB) blue nanoparticles to detect colorectal adenomas, a unique strain of C57BL/6J Min mice (adenomatous polyposis coli (Apc^{+/Min-FCCC}) was used that spontaneously develops multiple colorectal adenomas, as described in Cooper, H. S., et al. Mol. Carcinog. 2005, 44 (1), 31-41 and Clapper, M. L.; et al., Neoplasia 2011, 13 (8), 685-69, each of which is incorporated herein by reference in its entirety, herein. A breeding colony was established at Fox Chase Cancer Center (FCCC). Male and female mice (Apc^{+/Min-FCCC} and wild-type controls, n=5) enrolled in this study were maintained in a temperature—and humidity-controlled room. All animal protocols were approved by the Institutional Animal Care and Use Committee at FCCC. Mice bearing adenomas were injected retro-orbitally with a single dose of ZnPC(TB) loaded micelles (20 mg/kg based on the ZnPC(TB) dye). All mice tolerated the blue nanoparticle; no animal died or became moribund between the time of injection and the scheduled euthanasia. After injection, mice were dissected, and the colons were excised and opened their entire length. Twenty-four hours post-injection of ZnPC(TB)-blue nanoparticles into Apc^{+/Min-FCCC} mice revealed clear demarcation of tumor margins in the excised colon and small intestine tissues (even small tumors which are not visible by the naked eye), with efficient blue dye accumulation in polyps (FIG. 3A). The SBR for ZnPC(TB)-blue nanoparticle was lower at 0.52±0.15 compared to pre-injection SBR of 1.0±0.10, and 0.56±0.20 compared to pre-injection SBR of 1.0±0.14 with 20 mg/kg after 24 h injection for colons (Col) and small intestines (SI), respectively. Injection doses of less than 20 mg/kg showed inadequate accumulation of blue nanoparticles in tumor sites, especially in small intestines (FIGS. 7A-7D). In addition, it was seen that the (Apc)^{+/Min}-FCCC strain can develop stomach tumors too. It was found that ZnPC(TB)-blue nanoparticles can also mark stomach lesions (FIG. 3B).

Here, the amphiphilic block copolymers (PEG-PCL) were used as a carrier to deliver blue dyes to tumor sites. PEG-PCL can form micellar nanoparticles with a hydrophobic core and a hydrophilic shell. The hydrophobic core allows entrapment of agents with low aqueous dispersion, such as hydrophobic dyes, metals, and lipophilic drugs, while the hydrophilic shell can render aqueous dispersion and enhance colloidal stability of the polymeric nanoparticles. ZnPC(TB) blue nanoparticles preferentially accumulate in the tumor due to the enhanced permeation and retention (EPR) effect and positive surface charge. The visualization activity of phthalocyanine dyes:zinc, copper, nickel, and methylene blue was examined in a subcutaneous and orthotopic model, among which ZnPC(TB) blue nanoparticles showed the highest contrast activity. It was demonstrated that ZnPC(TB) blue nanoparticles could be used as a contrast agent to increase the detected number/margin of polyps in colorectal cancer.

Overall, results from the present study demonstrate that ZnPC(TB) loaded micelles identify colorectal adenomas, both nonpolypoid (flat) and polypoid, in Apc^{+/Min-FCCC} mice with high specificity. Both subcutaneous and orthotopic tumors could be identified using ZnPC(TB) blue nanoparticles. Blue nanoparticles increase the adenoma detection rate without increasing the removal of non-neoplastic lesions. ZnPC-loaded micelles are expected to enable high-contrast imaging of the mucosal surface in real-time without special optical imaging equipment (e.g., fluorescence, optical coherence tomography (OCT)), making it a quick and safe option for reducing the likelihood of follow-up surgeries and for improving overall survival. It is believed that the developed procedure will improve WLE and improve the contrast between malignant lesions and healthy tissue compared with chromoendoscopy. These studies should be validated by using an endoscope and large animal models in future work. This strategy offers precise delivery of blue dyes (i.e., tumors staining agents), which overcome the limitations of water-soluble dye spray during the endoscopy procedure.

### Conclusion

ZnPC nanoparticles were prepared (as described in Example 1) that enable high-contrast imaging of the mucosal surface without the use of special optical imaging equipment (e.g., fluorescence, OCT), making it a quick and safe option to improve the likelihood of a complete resection and reducing the need for follow-up surgeries.

### Example 3

### Evaluate the Visual Contrast Enhancement and Ability of Pc-Loaded Micelle to Enhance Intraoperative Image-Guided Resection in a Murine Model of Colorectal Cancer (CRC)

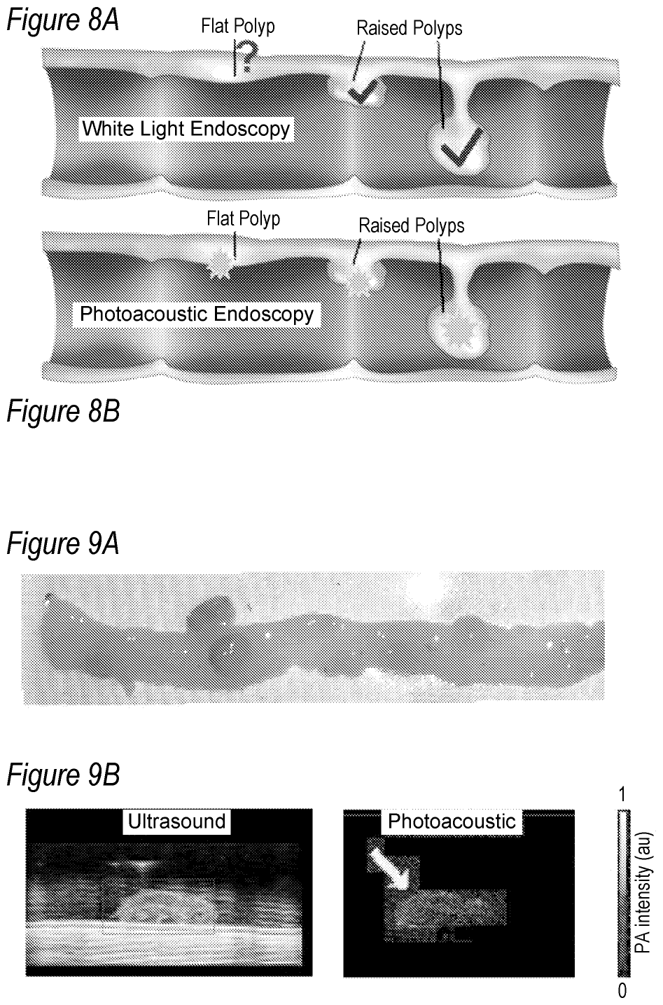

The most effective strategy for prevention of CRC is to screen for and remove precancerous polyps. Regular screening methods, e.g., White Light Endoscopy WLE), prevent 60% of deaths, and 9/10 of patients whose cancer is detected are alive 5 years later. Most polyps look something like a mushroom growing from the colon wall (i.e., raised polyps; FIG. 8A). They are easily seen during WLE and easily removed. But in high risk patients, current colonoscopy approaches, are insufficient to find flat polyps (FIG. 8A). Flat polyps, grow by spreading along the colon wall, are believed to make up about 9 percent of all polyps and be responsible for most of the colon cancers that occur in people who are up-to-date with their colonoscopies. So, there is a clear unmet need for sensitive and specific imaging approaches that enable detection of flat polyps in the CRC, ideally at a premalignant stage before invasive cancers develop (FIG. 8B).

### Techniques and Technology Assisted Detection

Targeted biopsy using topically applied dyes to delineate mucosal abnormalities (i.e., chromoendoscopy) has been shown to improve the adenoma detection rate by 30%.5 However, chromoendoscopy is not embraced by endoscopists due to the perceived hassle, cost, and time associated with intraluminal dye administration, and digital (image-enhanced) chromoendoscopy [e.g., narrow-band imaging and Fuji Intelligent Chromo Endoscopy (FICE)]have only shown marginally improved adenoma detection rates. Optical-guided surgery (e.g., photoacoustic imaging), has been gaining clinical acceptance over the last few years and has been used for the detection of lymph nodes, various tumor types, vital structures and tissue perfusion. The photoacoustic signal can be visualized in milliseconds, which is advantageous over other emerging imaging techniques, such as Raman spectroscopy and Optical Coherence Tomography, which require more time to visualize the same field of view. Also, in comparison to fluorescence imaging, photoacoustic imaging has improved sensitivity (˜4 times) for tumors deeper than 2 mm, the ability to image deeper seeded tumors (5-6 cm vs. 1 cm), and higher spatial resolution (˜100 μm). To mitigate the high miss rate (a miss rate of 11%-27% has been reported for WL detection of (pre) malignant colon lesions, particularly in high-risk patients, which compromises early detection and intervention) and low diagnostic accuracy of conventional WL endoscopy and negate the perceived drawbacks of chromoendoscopy, the aim is to improve early detection of (incipient) colorectal cancer during endoscopic surveillance using nanoparticle-based optical contrast agents. Imaging agents that highlight adenomas especially in high-risk cases, compatible with WLE (WLE needed for physiological context), do not interfere with colonoscopic workflow and do not add time. Owing to the favorable light transmission properties of photoacoustic imaging across biological tissue (in comparison to visible light), this modality can detect deep-seeded tumors and flat polyps and provide real-time navigation (FIG. 8B).

### Dye-Based Chromoendoscopy (DBC)

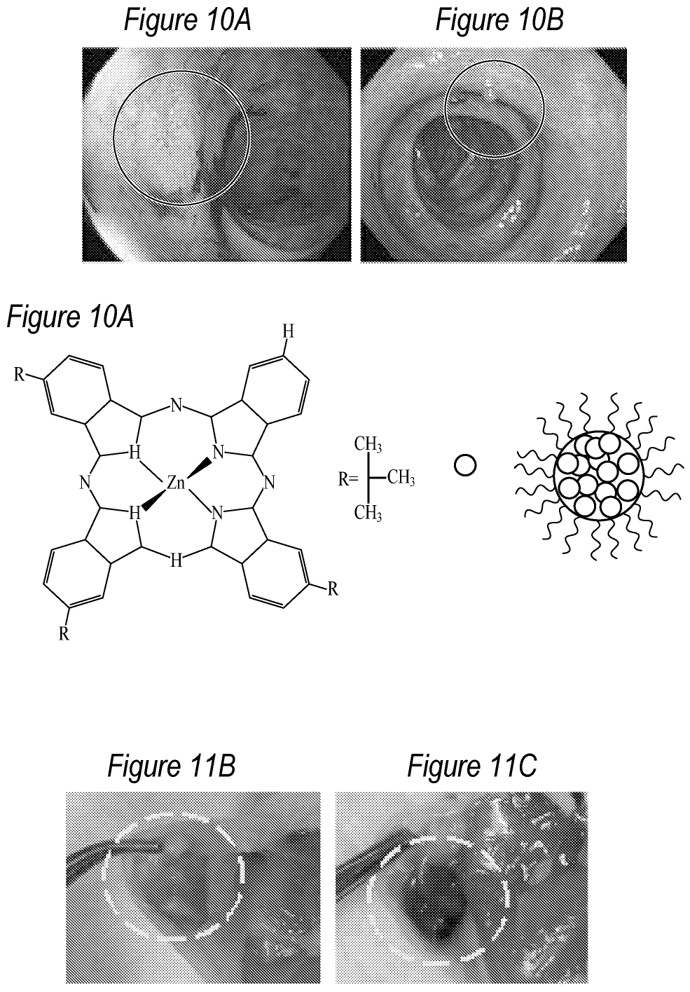

DBC has been introduced to improve patient outcomes, enabling the detection of dysplastic lesions in long-standing inflammatory bowel diseases (IBD). DBC involves applying a mucosal stain or dye (e.g., carmine or methylene blue), usually by injection down an endoscopic spray-catheter. However, DBC has some potential limitations hampering its feasibility in daily routine clinical practice. First, it is a time-consuming procedure, and the dye does not always coat the surface evenly, so distinguishing between cancerous (e.g., flat and/or polyp) and noncancerous tissue during resection is challenging (FIGS. 10A-1B), and the rate of missing tumor cells is high.

A tumor site is either a Flat lesion (FIG. 10A) or an inflammatory polyp (FIG. 10B) denoted with red circle. The disadvantage of the technique is the often-inhomogeneous staining pattern and distinguish between lesion/polyp, and healthy tissue is difficult (i.e., tumor margin is not clear). Furthermore, most gastroenterologists do not follow the DBC biopsy protocol frequently during surgery, as it is a time—and cost-expensive approach. Meanwhile, for intraoperative navigation, the surgeon has to rely primarily on visual and tactile feedback to distinguish between different kinds of tissue structures. Together with the increasing rate of minimally invasive (endoscopic/laparoscopic and robotic) surgery and, therefore, the lack of tactile feedback, there is a demand for improving the visibility of different kinds of tissue types, especially for distinguishing malignant and benign structures. However, classical stains or dyes (i.e., small molecules) lack sufficient tumor selectivity and are also taken up in healthy tissue, resulting in poor visibility of CRCs.

### Nanoparticle-Assisted Optical-Endoscopy

Polymeric micelles possess a supramolecular core-shell structure and represent a flexible nanoplatform for biomedical applications. Dye-loaded micelles (FIG. 1A) allow for the selective delivery of dyes into tumors, while minimizing the uptake in normal tissue, by the enhanced permeability and retention (EPR) effect. It is proposed to develop a new contrast agent for optical endoscopy that utilizes Pc dyes. Due to the high absorbance of Pc dyes at long wavelengths (λₘₐₓ>660 nm, εₘₐₓ>10⁵ Lmol⁻¹ cm⁻¹), and their strong photoacoustic activity, their encapsulation into nanoparticles make them a favorable choice compared with other optical contrast agents such as the commonly used indocyanine green (ICG) and methylene blue (MB) dyes, which are cleared within minutes upon injection. Pc-loaded micelles are expected to enable high-contrast photoacoustic imaging of the polyps in real-time, making it a quick and safe option for reducing the likelihood of follow-up surgeries and for improving overall survival. It is believed that the herein described developed procedures will improve early colorectal disease and flat polyps detection in high-risk patients, who undergo routine (e.g., annual) screening to monitor disease development or progression. In preliminary ex vivo studies in a resected open colon from an Adenomatous polyposis coli (Apc)^{+/Min} mice, photoacoustic imaging showed that the photoacoustic signal of the Pc-loaded micelle aligns well with polyps (FIG. 9D).

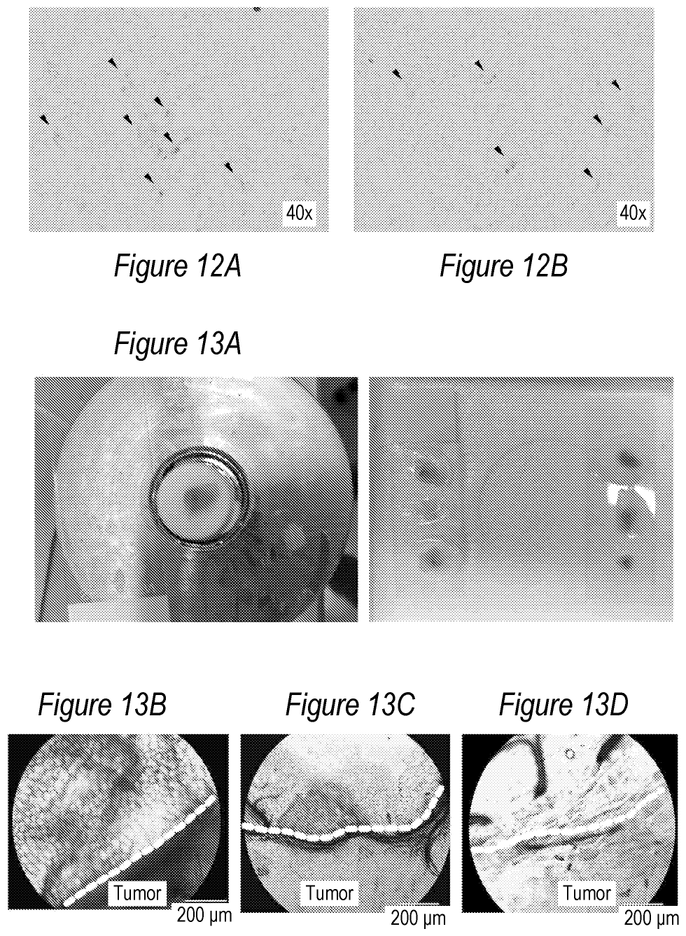

In particular, it is proposed to develop a new a new contrast agent for dye-based endoscopy that utilizes ZnPC dyes. ZnPC is a hydrophobic dye with an intense blue color. ZnPC-loaded micelles are expected to enable high-contrast imaging of the mucosal surface in real-time without the use of special optical imaging equipment (e.g. fluorescence, OCT), making it a quick and safe option for reducing the likelihood of follow-up surgeries and for improving overall survival. It is believed that the herein described and developed procedure will improve WLE and improve the contrast between malignant lesions and healthy tissue compared with chromoendoscopy. In preliminary studies in mice bearing 4T1 triple-negative breast tumors, found that ZnPC-loaded micelles could be delivered to the tumor site and significantly improve the ability to accurately identify tumor margins (FIG. 11A, 11B).

### 1. Synthesize and characterize the physical-chemical properties of Pc-loaded polymeric micelles

Various hydrophobic derivatives of Phthalocyanine (Pc) (e.g., with and without metal ions (M) and alkyl groups (R)) will be loaded into polymeric micelles, composed of the amphiphilic diblock copolymer PEG-PCL, via oil-in-water emulsions. The structural integrity of Pc-loaded micelles will be determined by transmission electron microscopy. The hydrodynamic diameter and zeta potential of Pc-loaded polymeric micelles will be measured by dynamic light scattering. The stability and Pc-release of polymeric micelles will also be evaluated as a function of time and pH in both storage and physiologic media.

Amphiphilic diblock copolymers (PEG-PCL) will be used to solubilize various hydrophobic derivatives of Pc into polymeric nanocarriers by using an established oil-in-water emulsion method. Size, charge, stability, and the extent of release of each Pc derivative from the respective micelle formulation, under different storage and physiological conditions, will be measured.

### 2. Evaluate Pc-Loaded Micelle In Vitro.

The cytotoxicity and uptake of Pc-loaded micelles will be evaluated in vitro as a function of dose and incubation time.

Cytotoxicity will be determined at 24, and 48 hours following the addition of various concentration of the Pc-loaded micelles on colon cancer cells (HT-29), endothelial cells (HUVEC), and liver cells (Hep G2) via cell proliferation (MTT) and apoptosis (caspase-Glo, Promega) assays. The uptake of micelles by HT-29 will also be evaluated.

### Evaluate Pc-Loaded Micelle In Vivo by Intraoperative Optical Endoscopy.

The optimal micelle formulation identified from studies in parts 1 and 2, based on high stability, low leakage, and low cytotoxicity, will be tested in a limited number of mice as a pilot study. Apc^{+/Min} mice that spontaneously develop multiple colorectal adenomas will be injected (i.v.) with Pc-loaded micelles, and the colon will be imaged via photoacoustic imaging (1-day post injection) and then ex vivo visualization of intestinal adenomas will be performed. Image analyses will be correlated with histopathologic findings for all regions of interest (ROIs).

The optimal micelle formulation that is found in the synthesis and evaluation of Pc-loaded micelle in vitro with high stability and low cytotoxicity will be imaged in a limited number of mice that spontaneously develop multiple intestinal neoplasia (Min) via photoacoustic imaging. The tumor-to-background contrast and the accuracy of tumor margin identification will be quantified.

Summary of Anticipated Results and their Utility for Researcher's Long-Term Goals

Anticipated results: 1) prepare reproducible, stable, and biodegradable Pc-loaded micelles with high drug encapsulation efficiency (>85%), low leakage (<10% in 24 hrs in serum) and narrow-size distribution (PDI<0.2). 2) demonstrate that Pc-loaded micelles exhibit significant improvement in raised and flat tumors visualization and high accuracy, using histological assessments for validation. Our long-term goal is to develop a new nano-based optical contrast agent for more effective deep-sited tumor visualization. It is believed that this innovative nanotechnology will help to identify (flat) lesions during intraoperative procedures and profoundly improve the ability of physicians to completely eradicate the local disease in endoscopy and/or laparoscopic surgical procedures.

### 4. Synthesize and characterize the physical-chemical properties of ZnPC-loaded polymeric micelles

Various hydrophobic derivatives (e.g., with and without alkyl groups) of the ‘blue’ hydrophobic dye Zinc Phthalocyanine (ZnPC) will be loaded into polymeric micelles, composed of the amphiphilic diblock copolymer PEG-PCL, via oil-in-water emulsions. The structural integrity of ZnPC-loaded micelles will be determined by transmission electron microscopy. The hydrodynamic diameter and zeta potential of ZnPC-loaded polymeric micelles will be measured by dynamic light scattering. The stability and ZnPC-release of polymeric micelles will also be evaluated as a function of time and pH in both storage and physiologic media.

### 5. Evaluate ZnPC-Loaded Micelle In Vitro.

Cytotoxicity will be determined at 24, and 48 hours following the addition of various concentration of the ZnPC-loaded micelles on colon cancer cells (HT-29), endothelial cells (HUVEC), and liver cells (Hep G2) via cell proliferation (MTT) and apoptosis (caspase-Glo, Promega) assays. The uptake of micelles will also be evaluated.

### 6. Evaluate ZnPC-Loaded Micelle In Vivo by Intraoperative Endoscopy.

The optimal micelle formulation identified from studies in parts 4 and 5, based on high stability, low leakage, and low cytotoxicity, will be tested in a limited number of mice as a pilot study. Adenomatous polyposis coli (Apc)+/Min mice that spontaneously develop multiple colorectal adenomas will be injected (i.v.) with ZnPC-loaded micelles, and the colon will be imaged via WLE (1-d post injection) and then ex vivo visualization of intestinal adenomas will be performed. Image analyses will be correlated with histopathologic findings for all regions of interest (ROIs).

Summary of Anticipated Results and their Utility for Researcher's Long-Term Goals

Anticipated results: 1) prepare reproducible, stable, and biodegradable ZnPC-loaded micelles with high loading capacity (>85% efficiency), low leakage (<10% in 24 hrs in serum) and narrow-size distribution (PDI<0.2). 2) demonstrate that ZnPC-loaded micelles exhibit significant improvement in tumor margin visualization and high accuracy, using histological assessments for validation. Our long-term goal is to develop a new imaging agent for more effective tumor visualization. It is believed that this innovative nanotechnology will help to identify lesions during intraoperative procedures and profoundly improve the ability of physicians to completely eradicate the local disease in endoscopy and/or laparoscopic surgical procedures.

### Example 4

Evaluate the Contrast Enhancement and Ability of Pc-NP to Enhance Intraoperative Image-Guided Surgery and Treatment in a Murine Model of Ovarian Cancer Techniques and technology assist detection and treatment Solid tumors are often treated by surgery and post-surgical chemo—and radiotherapies. However, precise targeting and real-time intraoperative localization of the tumor margins remains a challenging task, which usually results in tumor recurrence if residual tumor is left within the resected regions. Therefore, it is necessary to resect larger areas of surrounding normal tissues or even whole organ in order to minimize the chances of recurrence.

Computed tomography (CT), magnetic resonance imaging (MRI), positron emission tomography (PET), single-photon emission computed tomography (SPECT), and ultrasonography all have unique advantages in visualizing tumors, and technical improvements such as multimodality systems (PET-CT, PET-MRI) have led to increasing efficiency and sensitivity and detailed two—and three dimensional images. However, these modalities are not suitable for real-time feedback during surgery. In diseases with a peritoneal spreading pattern, such as ovarian cancer, cytoreduction is of eminent importance. Current methods of intraoperative margin assessment include frozen section, imprint cytology, intraoperative ultrasound, wire localization, and radioguided localization. These methods are labor intensive, time consuming, may lead to poor cosmesis, and in some cases are limited in their ability to assess the entire margin. In contrast, co-registered photoacoustic (PA) and ultrasound (US) imaging is a real time and affordable technique. The photoacoustic signal can be visualized in milliseconds, which is advantageous over other emerging imaging techniques, such as Raman spectroscopy and Optical Coherence Tomography, which require more time to visualize the same field of view. PA imaging produces a tomographic image in vivo with very high spatial resolution (up to 50-500 μm) and depth of penetration up to 5 cm.¹

In terms of ovarian cancer treatment, although conventional treatments, including surgery, chemotherapy, radiotherapy, etc., have proven to be efficient, they are often associated with certain unignorable weaknesses, such as immune system depletion, high cost, patient nonadherence, tumor recurrence and drug resistance. These clinical problems have accordingly heightened the need for more specific and effective tumor treatment. A recent trend in more aggressive cancer therapies involves combining surgical resection with phototherapy. Clinical studies have shown that overall survival is largely improved in patients treated with phototherapy as an adjunct to surgery. Benefits of image guided surgery and phototherapy have been shown. The phthalocyanine (Pc) is organic dyes that have been explored widely for optical imaging and phototherapy, exhibit good photothermal conversion efficiency and are capable of producing both a photodynamic therapy (PDT) and photothermal therapy (PTT) effect. Owing to their strong absorption within the near-infrared (NIR) window, Pc derivatives have shown significant potential as theranostic agents. Several Pc derivatives have either already been through the FDA approval process or are under clinical trials. However, PC derivatives are small hydrophobic dyes that generally exhibit poor solubility, thereby limiting dose, and rapid clearance, which limits tumor accumulation. To improve their clinical efficacy, our goal is two-fold. First, a small library of PC derivatives will be screened to identify the compound that exhibits the strongest PA contrast and therapeutic efficacy as a PDT/PTT agent. It was found that even just small structural changes to PC can have a profound impact on their optical and therapeutic properties. Second, the PC variants will be formulated into a novel theranostic nanomedicine platform that will improve PC circulation time and biodistribution. It is hypothesized that improved pharmacokinetics will improve the identification of tumor margins and metastases and will enable the more effective treatment of unresected and residual lesions, via intraoperative multimodal phototherapy. It has previously been shown that a synergistic effect of combined noninvasive PDT and PTT therapy can also overcome multidrug resistance and decrease the dosage-limiting toxicity of current chemotherapy drugs. Nanoparticle-assisted optical-imaging and treatment

Polymeric micelles possess a supramolecular core-shell structure and represent a flexible nanoplatform for biomedical applications. Dye-loaded micelles (FIGS. 1A and 1B(i)-1B(ii)) allow for the selective delivery of dyes into tumors, while minimizing the uptake in normal tissue, by the enhanced permeability and retention (EPR) effect. It is proposed to develop a new theranostic agents for photoacoustic imaging and phototherapy that utilizes Pc dyes. Pc-loaded micelles are expected to enable high-contrast intraoperative imaging of ovarian cancers in real-time and treatment of residual lesions, making it a quick and safe option for reducing the likelihood of follow-up surgeries and for improving overall survival. It is believed that the herein described and developed procedure will improve the identification of tumor margins and metastases, thus improving the likelihood of a complete resection, and provide an additional PDT/PTT treatment option for the elimination of any residual disease. In preliminary in vivo studies in a subcutaneous xenograft of the 4T1 breast cancer and a resected open colon from an Adenomatous polyposis coli (Apc)+/Min mice, photoacoustic imaging showed that the photoacoustic signal of Pc-loaded micelles align well with breast (FIG. 1D) and colon tumors (FIG. 1F). In addition, a temperature increase, of ΔT˜32° C. was observed for NiPC— and ZnPC-loaded nanoparticles in water under laser irradiation (FIG. 1F). Typically, an increase of just 5° C. for 10 min is sufficient for cell killing by PTT. Therefore, it is believed that C-loaded micelles can serve as an effective phototherapeutic agent. 1. Synthesize and characterize the physical-chemical properties of Pc-loaded polymeric micelles.

Commercially available hydrophobic derivatives of PC (with and without metal ions (M) and alkyl groups(R)) will be loaded into polymeric micelles, composed of the amphiphilic diblock copolymer PEG-PCL, via oil-in-water emulsions. While PC variants are structurally similar, their photoacoustic and PTT properties can vary significantly. Therefore, micelles will be prepared with all derivatives of Phthalocyanine (PC) dyes and/or naphthalocyanine (NC) dyes, which are described hereinabove in the Detailed Description of the Invention, and their physical-chemical properties will be characterized. The structural integrity of Pc-NP will be determined by transmission electron microscopy. The hydrodynamic diameter and zeta potential of Pc-loaded polymeric micelles will be measured by dynamic light scattering. The stability and Pc-release of polymeric micelles will also be evaluated as a function of time and pH in both storage and physiologic media. The photothermal conversion coefficient and reactive oxygen species (ROS) generation will be investigated.

### 2. Evaluate Pc-Loaded Micelle In Vitro.

Photocytotoxicity will be determined as a function of Pc-loaded micelle dose, as well as laser power, exposure time and frequency of exposure. An 808 nm laser will be used for all cell studies, since it is already widely utilized in a clinical setting for photothermal ablation. Cytotoxicity studies will be performed on ovarian cancer cells ID8 and SKOV3 and quantified via a cell proliferation assay (MTT). The uptake of micelles will also be evaluated via confocal microscopy.

### 3. Evaluate Pc-Loaded Micelle In Vivo by Intraoperative Optical Imaging and Treatment.

The optimal micelle formulation identified from studies in parts 1 and 2, based on high stability, low leakage, and low cytotoxicity, high photoacoustic signal, and high phototoxicity will be tested in a limited number of mice as two pilot studies. The first pilot study will be conducted to detect tumor margins in subcutaneous model and then partial resection under PA image-guided surgery. Any remaining residual tumors will be ablated by using post-surgical adjuvant photothermal therapy. A second pilot study will be performed to identify tumor margins via PA imaging in an orthotopic model of ovarian cancer, followed by photothermal ablation. Ovarian (SKOV3) tumor-bearing mice will be injected (i.e., intravenously) with Pc-NP. PA imaging will be run immediately prior to injection and 24 hr post-injection to assess Pc-NP accumulation within the tumor, a partial resection will be performed and then the unresected tumor will be exposed to 808 nm laser to eliminate any residual cancer cells. In the ovarian orthotopic model (SKOV3 (Luc+)), tumor progression will be monitored by using bioluminescent imaging. Tumors will be resected with wide margins and sectioned. The distance between the Pc-NPs and the true tumor, margin as identified in histopathology, will be quantified.

Summary of Anticipated Results and their Utility for Long-Term Goals

Anticipated results: 1) prepare reproducible, stable, and biodegradable Pc-loaded micelles with high loading capacity (>85% efficiency), low leakage (<10% in 24 hours in serum) and narrow-size distribution (PDI<0.2). 2) demonstrate that Pc-loaded micelles exhibit significant improvement in ovarian cancer detection/resection and high accuracy and eliminate tumors via PTT/PDT. The long-term goal is to develop a new nano-based theranostic agent for more effective identification of tumor margins and metastases and treatment of residual invasive disease. It is believed that this innovative nanotechnology will profoundly improve the ability of physicians to completely eradicate local disease in ovarian cancer patients, resulting in fewer re-excisions and ultimately leading to an improvement in progression-free and overall survival.

### Example 5A

### Phthalocyanine-Blue Nanoparticles for Detection of Colorectal Cancer In Vivo Studies

This study sought to develop phthalocyanine (PC) dye-loaded micelles, that could enable the direct visualization of colorectal tumors and colon polyps with the naked eye. Owing to their dark blue or green color, it was hypothesized that accumulation of PC-loaded micelles within tumors via the EPR effect would lead to discoloration of the tumors and facilitate their identification. PCs have been explored previously for various biomedical applications, including photodynamic therapy, photothermal therapy, and optical imaging with several PCs already in clinical trials. Therefore, this class of dyes is considered biocompatible and safe; although this will need to be independently verified for each unique dye molecule. Six PC dyes (PC, Zinc PC, Copper (II) PC, Zinc PC (Tetra-Tert-Butyl(TB)), Nickel PC, and Zinc PC (octakire evaluated. Each dye was encapsulated within micelles that were formed using the amphiphilic diblock copolymer polyethylene glycol-polycaprolactone (PEG-PCL) (FIGS. 1A, 1B). The chemical structures and complete chemical names of the phthalocyanine dyes used in this study are provided in FIGS. 4A-4F. Because of their hydrophobic nature, PCs tend to aggregate (π-π stacking) when dissolved directly in aqueous media; however, they are readily solubilized upon encapsulation in micelles and appear as transparent, colored solutions (FIG. 1B, FIG. 5A). The median hydrodynamic diameter of the various PC-loaded micelles was 50 to 100 nm, as determined by dynamic light scattering (DLS), with an approximate polydispersity index (PDI) of 0.2 (FIG. 1B). All of the micelles exhibited a negative zeta potential between −15 to −25 mV, except for the ZnPC(TB)-loaded micelles, which exhibited a slightly-positive zeta potential (˜5 mV) (FIG. 1B). UV-visible spectroscopy showed a strong absorbance peak for the respective PC dyes, with peak absorbances between 600 and 850 nm (FIG. 5B). The release of the PC dyes from the PC-loaded micelles was assessed in sodium cholate buffer as a function of time, via a dialysis method.30 As shown in FIG. 5C, all of the PCs demonstrated high retention within micelles after dialysis (>95% in 24 h), likely because of their hydrophobicity, which is indicated by their positive octanol-water partition coefficient (log P values, estimated with the ALOGPS algorithm 31) (FIG. 5C). All but CuPC possessed a log P value greater than 5. In contrast, methylene blue (MB), which is a highly water-soluble dye, possessed a negative log P and was released entirely from the dialysis tubing.

To identify the PC-loaded micelle formulation that is best able to differentiate tumors from surrounding healthy tissues via direct visualization, BALB/c mice bearing syngeneic 4T1 triple-negative breast flank tumors were injected intravenously with the various micelle formulations (10 mg/kg based on the dye concentration, 24 h before evaluation). The ZnPC(TB)-loaded micelles clearly led to the greatest contrast enhancement, with the tumors turning a very dark blue color, while the other micelle formulations only led to a faint discoloration (FIG. 1C). For a comparison, methylene blue (MB), a clinically approved water-soluble blue dye, was also injected intravenously, but no accumulation was observed at the tumor sites. Normalized signal-to-background (SBR) measurements were calculated in ImageJ using the intensity of the blue color within the tumor and its surrounding tissue, respectively.

The SBR of tumors from mice injected with ZnPC(TB)-loaded micelles was significantly lower at 0.28±0.05, compared to tumors from mice injected with water, which possessed an SBR of 1.0±0.09 24 h after injection (FIG. 6B). These results indicate that ZnPC(TB)-loaded micelles are a suitable candidate for tumor visualization. At a dose of 10 mg/kg ZnPC(TB)-loaded micelles, some discoloration of the skin surrounding the tumor was observed; however, this was not seen upon surgical exposure of the tumor, through the creation of a skin flap (FIG. 6A). The discoloration was reduced when the micelle dose was decreased to 5 mg/kg, but the tumor contrast was also reduced (FIG. 6B).

It is postulated that the improved accumulation of the ZnPC(TB)-loaded micelles within tumors may stem from its slightly-positive zeta potential, since all of the micelle formulations tested otherwise possess similar physical-chemical properties. A positive surface charge could be responsible for significant differences in the opsonization of serum proteins, which could impact biodistribution. A positive surface charge could also facilitate electrostatic interaction towards negatively charged proteoglycans located within the plasma membrane of cells.

After identifying ZnPC(TB)-loaded micelles as our lead formulation, we conducted a more thorough evaluation on the influence of synthetic conditions on the physical-chemical properties of the micelle. In particular, the effect of varying the amount of ZnPC(TB) dye, with a fixed amount of PEG-PCL, on the physical-chemical properties of the ZnPC(TB)-loaded micelles was evaluated. Encapsulation efficiency was >95% for ZnPC(TB) when the Zn(TB):PEG-PCL ratio (w/w) was in the range of 0.25:1 to 1:1, but dropped off significantly as the ratio of dye-to-polymer was increased further (Table 2). The ZnPC(TB) payload was approximately 48% of the total weight (ZnPC(TB)+PEG-PCL) at 1:1 dye-to-polymer ratio. In addition, the hydrodynamic diameter increased from −35 nm to −120 nm as the ZnPC(TB):PEG-PCL ratio increased. ZnPC(TB)-loaded micelles formed using a ZnPC(TB):PEG-PCL ratio of 0.5:1 were used for all subsequent studies, since they provided a favorable balance between small size, high drug payload, and showed favorable results that we already acquired in the preliminary animal studies. While using micelles formed at a ZnPC(TB):PEG-PCL ratio of 1:1 was considered, there was a concern that reproducibility would be compromised, due to dramatic changes in size and encapsulation, with any further increase in the ZnPC(TB):PEG-PCL ratio. The preparation of ZnPC(TB)-loaded micelles at a ZnPC(TB):PEG-PCL ratio of 0.5:1 was highly reproducible and easily scalable, with <5% variation in size and encapsulation efficiency and similar zeta potential, from batch-to-batch (Table 3). The ZnPC(TB)-loaded micelles demonstrated size stability during storage in PBS without aggregation or precipitation, as indicated by no significant change in hydrodynamic diameter for at least 7 days (FIG. 2B).

TABLE 2
Physical-chemical properties of ZnPC (TB) loaded micelles.

| ZnPC (TB): PEG-PCL Ratio | | | | | | |
|---|---|---|---|---|---|---|
| (w/w) | 0.25:1 | 0.5:1 | 1:1 | 2:1 | 4:1 | 20:1 |
| Encapsulation Efficiency (%) | 96.0 | 97.2 | 97.3 | 68.7 | 32.1 | 7.3 |
| Payload (%) | 19.2 | 32.4 | 48.7 | 45.8 | 25.68 | 6.95 |
| Hydrodynamic Diameter (nm) | 37 | 48 | 59 | 107 | 110 | 119 |
| Polydispersity Index | 0.215 | 0.175 | 0.195 | 0.234 | 0.180 | 0.265 |

TABLE 3
Repeatability of ZnPC (TB) loaded micelle synthesis (ratio 0.5:1)

| | Batch 1 | Batch 2 | Batch 3 | Average | St. Dev |
|---|---|---|---|---|---|
| Hydrodynamic | 47 | 55 | 53 | 51.6 | 3.39 |
| Diameter (nm) | | | | | |
| PDI | 0.187 | 0.212 | 0.193 | 0.197 | 0.0106 |
| Encapsulation | 98.1 | 95.4 | 97.5 | 97 | 1.15 |
| Efficiency (%) | | | | | |
| Zeta-Potential (mV) | 2 | 11 | 5 | 6 | 3.7 |

The cytotoxicity of the ZnPC(TB) nanoparticles was examined in an MTS cell proliferation assay. Increasing concentrations of ZnPC(TB)-loaded micelles were incubated with 4T1 murine breast cancer cells or human colon cancer (HT-29) cells for 24 h. ZnPC(TB)-loaded micelles exhibited no obvious cytotoxicity in either cell type at concentrations up to 100 μg/mL (FIG. 2C).

To assess the feasibility of using the ZnPC(TB)-loaded micelles to detect colorectal adenomas, we used C57BL/6J Min mice (adenomatous polyposis coli (Apc^{+/Min-FCCC})), which spontaneously develop multiple colorectal adenomas. Mice bearing adenomas (n=5) were injected retro-orbitally with a single dose of ZnPC(TB)-loaded micelles (20 mg/kg based on the ZnPC(TB) dye). Sterile water was used for control mice (n=5). All mice tolerated the micelles; no animals died or became moribund between the time of injection and the scheduled euthanasia. Twenty-four hours after injection, the small intestine and colons were excised and cut open along their entire length.

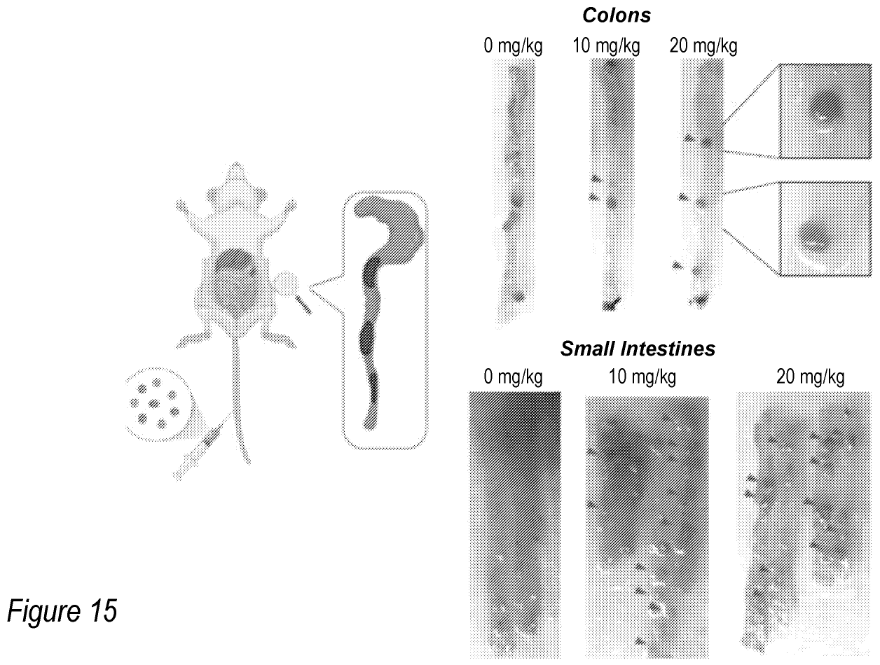

The excised colon and small intestine tissues from Apc^{+/Min-FCCC} mice that received ZnPC(TB)-loaded micelles revealed clear demarcation of tumor sites, which appeared blue. Even small tumors, which are not visible to the naked eye, could easily be identified with appreciable blue discoloration (FIG. 3A). The SBR of tumors within the colon (Col) and small intestine (SI) in mice injected with ZnPC(TB)-loaded micelles was 0.52±0.15 and 0.56±0.20, respectively, as compared to those of water-injection controls (SBR of 1.0±0.10 for Col and 1.0±0.14 for SI). See FIG. 15) The ZnPC(TB)-loaded micelles were also able to identify stomach lesions, which can develop in Apc^{+/Min-FCCC} mice (FIG. 3B). Injection doses of less than 20 mg/kg showed inadequate micelle accumulation to clearly identify all of the tumor sites, especially in the small intestine (FIGS. 7A-7D).

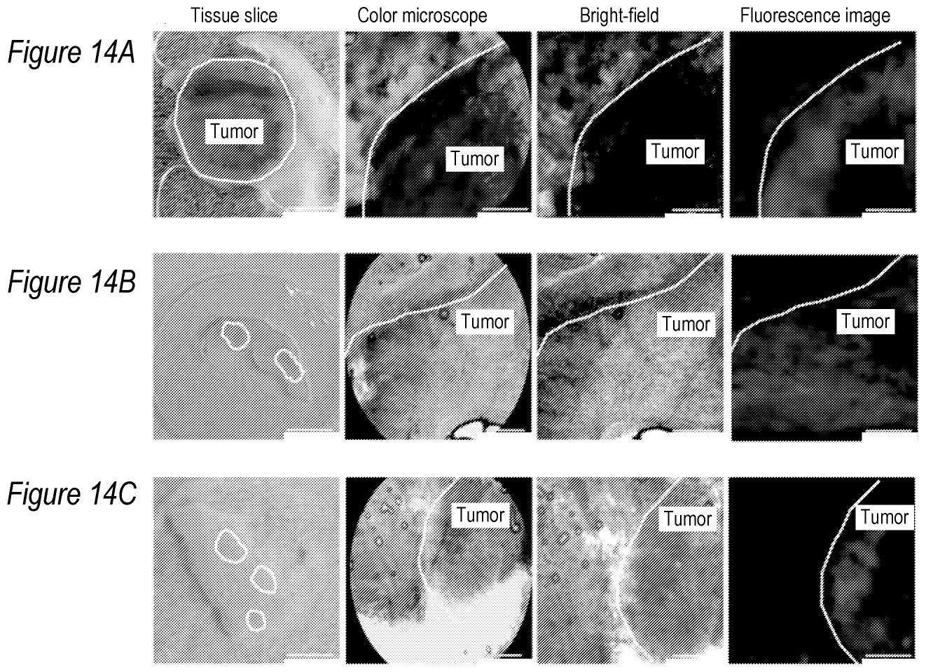

Routine histopathology was used for the final assessment of tissue samples. A significant challenge of this work was the loss of the blue color during tissue processing. Frozen sections (10 μm-thick) exhibited only a scattering of faint blue staining (FIGS. 12A-12B) and hematoxylin and eosin-staining led to complete loss of the blue color (data not shown). In an attempt to retain more of the blue coloring during tissue processing, frozen tissues embedded in optimal cutting temperature compound (OCT) were sectioned at an average cut depth of ˜0.5-1 mm using a hand microtome (FIG. 13A). Tissue sections were transferred to glass slides and imaged. Color, bright-field, and fluorescence images of flank, colon, and small intestine tumor tissues are shown in FIGS. 14A-14C, respectively. The open-source image processing package Fiji was used to process the tissue images. While much of the blue color was still faded, especially for the colon and small intestine tissue samples, some blue-green color can still be observed within all of the tumors. In the flank breast tumors, the tumor margin is clearly demarcated (FIG. 14A and FIGS. 13B-13D). In the colon and small intestines, where the blue color is less visible because of the tissue processing, the presence of the ZnPC(TB)-loaded micelles can still be ascertained by fluorescence imaging. In all of the tissue samples that were analyzed, the fluorescent signals closely align with the tumor margin, which is outlined with a white-dashed line and can generally be identified on the color images by a high nuclear density and poorly differentiated cells.

Overall, results from the present study demonstrate that ZnPC(TB)-loaded micelles can be used to identify syngeneic breast tumors as well as colorectal adenomas. Polypoid lesions were easily identified in Apc^{+/Min-FCCC} mice with high specificity. The ZnPC(TB)-loaded micelles are expected to enable high-contrast imaging of the mucosal surface in real-time without special optical imaging equipment (e.g., fluorescence, optical coherence tomography), making it a quick and safe option for reducing the likelihood of follow-up surgeries and for improving overall survival. It is believed that ZnPC(TB)-loaded micelles could improve WLE and enhance the contrast between malignant lesions and healthy tissue, as compared to chromoendoscopy. This strategy offers precise delivery of blue dyes (i.e., tumor staining agents), which overcome the limitations of water-soluble dye sprays during the endoscopy procedure. Although the results of these studies need to be validated in the future using an endoscope and large animal models, preliminary findings are encouraging. More precise techniques to assess the accuracy and precision of the ZnPC(TB)-loaded micelles in identifying the tumor margin must also still be developed. Nonetheless, this work presents a new contrast agent and approach that could potentially enable clinicians to more effectively clear the colonic polyps and reduce the likelihood of occult lesions that can grow and progress into more threatening processes.

### Example 5B

### Phthalocyanine-Blue Nanoparticles for Detection of Colorectal Cancer

### METHODS Materials

Methylene blue (MB), Zinc 2,3,9,10,16,17,23,24-octakis(octyloxy)-29H,31H-phthalocyanine (ZnPC(octakis)), Nickel(II) 1,4,8,11,15,18,22,25-octabutoxy-29H,31H-phthalocyanine (NiPC(octa)), Copper(II) phthalocyanine (CuPC), Zinc phthalocyanine (ZnPC), Zinc 2,9,16,23-tetra-tert-butyl-29H,31H-phthalocyanine (ZnPC(TB)) were purchased from Sigma, and 29H,31HPhthalocyanine (PC) was obtained from Santa Cruz Biotechnology. Poly (ethylene glycol)-co-poly (caprolactone) (PEG-PCL) was obtained from Polymer Source Inc. Preparation of PCs nanoparticles: A mixture (200 μL) containing PCs dyes (2 mg into dimethyl sulfoxide (DMSO)) and amphiphilic diblock copolymer poly(ethylene glycol)-poly(ε-caprolactone) (PEG-PCL) (4 mg in toluene) was quickly added into a glass vial containing 4 mL of deionized water under sonication at room temperature until a homogeneous solution was observed. The toluene was evaporated overnight. Dialysis was performed with dialysis tubing (3500 MWCO) into 4L of deionized water to remove DMSO.

### Characterization of PCs Nanoparticles

The diameter and size distributions of the PCs nanoparticles were measured with dynamic light scattering (DLS, Malvern, Zetasizer, Nano-ZS). The encapsulation efficiency and payload of PCdyes were determined using a UV-Vis spectrophotometer (Varian, 100 Bio).

### PCs Nanoparticles Retention Studies

100 μL of PCs nanoparticles was diluted in 3 mL of 20 mM sodium cholate solution. Dialysis was performed with dialysis tubing (12,000-14,000 MWCO, Fisher) into 500 mL of 20 mM sodium 3cholate buffer at room temperature. The buffer was changed after 4 hours. The absorbance of the solutions was recorded before and after (24 hours) dialysis to determine dye retention percentage.

### MTS Assay

4T1 (triple-negative breast cancer) and HT-29 (colon cancer) cells (1×104 cells per well) were seeded in 96-well plates and incubated overnight to allow the cells to attach to the surface of the wells. The cells were then mixed with increasing concentrations of ZnPC (TB) blue nanoparticles for 24 h. According to the supplier's instructions, the cell viabilities were determined using an MTS assay (Abcam Inc.). Briefly, after 24 h of incubation with ZnPC(TB) blue nanoparticles, 10ul of MTS reagent was added. After 2 h, absorbance was read at 490-nm on a Tecan microplatereader.

### In Vivo Studies

4T1 tumor cells (2×10⁶ cells in 100 ul) were implanted in the right flank of BALB/c mice (aged 6 weeks). When the tumor size reached approximately 100 mm³, nanoparticles (5 and 10 mg/kg) were injected into the mice via retro-orbital injection. At 24 h after injection, all animals were anesthetized according to an approved Institutional Animal Care and Use Committee (IACUC) protocol and euthanized by CO₂ inhalation/cervical dislocation. Tumor tissues were collected for further evaluation.

Male Apc^{+/Min-FCCC} (C57BL/6J) mice (120-150 days old) were obtained from an established breeding colony at Fox Chase Cancer Center (FCCC). Mice were genotyped for a point mutation in codon 850 of the Apc gene according to an established protocol. Mice were maintained in a temperature—and humidity-controlled room and received Harlan Teklad 2018 diet (Harlan Teklad, Madison, WI) and drinking water ad libitum. All animal protocols were approved by the Institutional Animal Care and Use Committee at FCCC. Mice were injected retro-orbitally with the ZnPC(TB)-loaded micelles (<150 μL). ZnPC(TB)-loaded micelles were filtered with a 0.2 m filter before use. Twenty-four hours after injections, mice were euthanized. The small intestine and colon were excised and grossly examined to determine the location of nanoparticles.

### Blue Dye Accumulation (Tissue Evaluation)

To assess blue nanoparticle accumulation in the breast and spontaneous colorectal mice model, tumors were evaluated visually, and normalized signal-to-background (SBR) was quantitated for each tumor relative to the surrounding tissue by measuring the intensity of blue-green colors via ImageJ. In addition, to evaluate tumor tissues, tumors (i.e., breast and spontaneous colorectal) were collected from mice at 24 h after injection (water and micelle) and stored in 10% neutral buffered formalin solution or dry ice. Optimal cutting temperature compound (OCT compound) was used to embed tumor tissue samples before frozen sectioning on a hand microtome. Tissues were covered in OCT compound (with no air bubbles in it) into a plastic mould, and then the mould was immersed gently in liquid nitrogen. Tissues were cut around 0.5-1 mm thick. Tissues were placed in microscope glass slides, imaged fluorescence in the Cy5 filter (excitation 628-40 nm, emission 692-40 nm), and analyzed via ImageJ. The unpaired t-test was used for analyses (P-value). *P, **P, and ***P<0.001. Statistical significance was defined as P<0.05.

### Example 6

### Phthalocyanine and Naphthalocyanine Loaded Micelles for Photoacoustic Image-Guided Surgery and Photothermal Therapy

The fifth leading cause of cancer mortality in women is ovarian cancer. Debulking is the best way to lower the risk of recurrence and improve the effectiveness of adjuvant chemotherapy. Unfortunately, optimal cytoreduction remains challenging due to residual microscopic tumors that could lead to relapse. To overcome this issue, developing an imaging and therapeutic approach that improves complete tumor resection, metastasis detection, and treatment of remaining disease could extend the length of progress-free survival. Current intraoperative margin assessments are limited in their ability to localize to the tumor and assess the entire margin in real-time. Comparatively, photoacoustic imaging (PA) has high contrast and spatial resolution, low cost, and provides real-time imaging. This can be combined with photothermal therapy (PTT), which has been shown to significantly improve overall survival in clinical trials. PTT is highly specific, has low toxicity to normal tissue, and is oxygen-independent. Napthalocyanines (Ncs) and Phthalocyanines (Pcs) dyes have proven effective for both PTT and PA with good photothermal stability, but they are highly hydrophobic and aggregate under physiological conditions. To improve the solubility of hydrophobic Pcs and Ncs, they were loaded into biocompatible and biodegradable poly (ethylene glycol)-co-poly (caprolactone) (PEG-PCL) micelles and employed as both a contrast agent for photoacoustic imaging and a photothermal therapy agent.

### Material & Methods

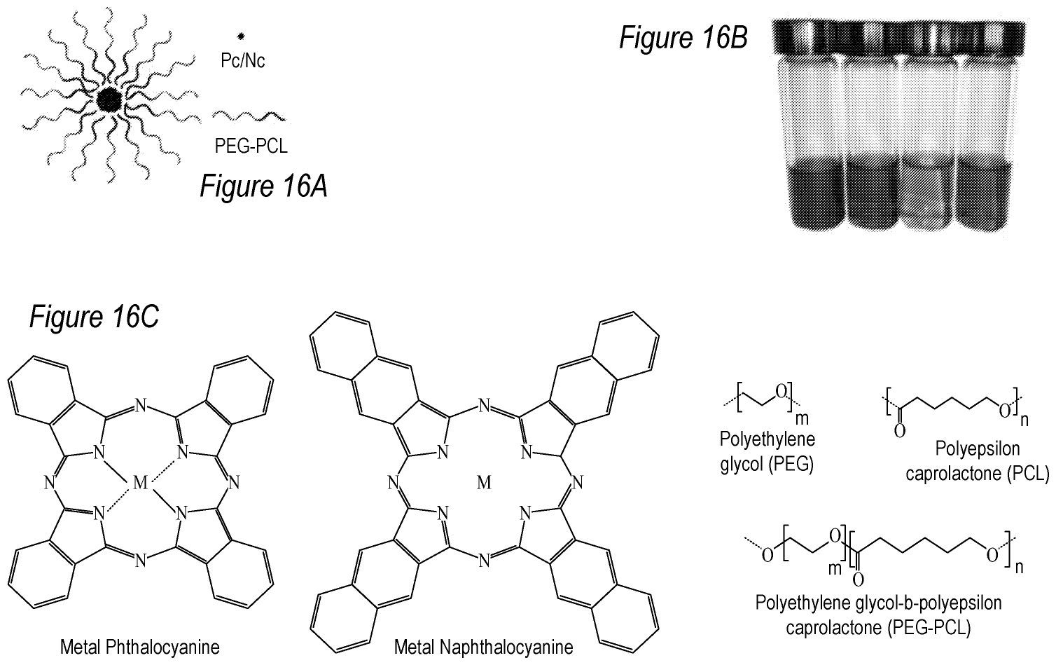

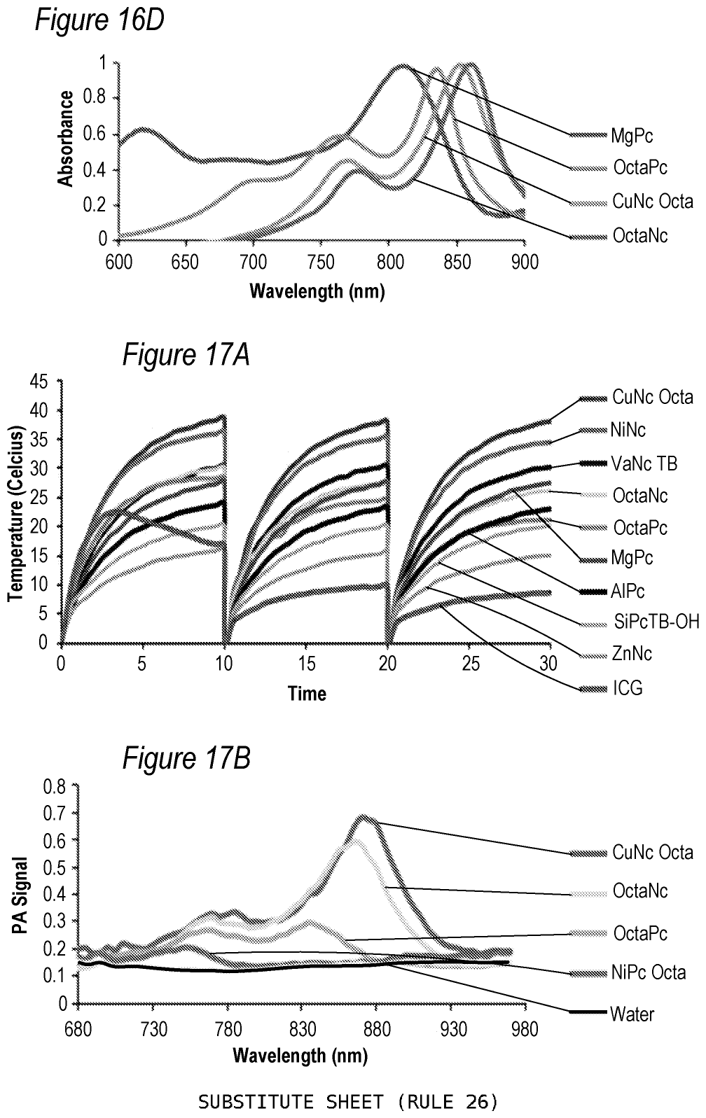

A total of twenty-eight phthalocyanine and nine naphthalocyanine dyes were loaded into polymeric micelles, composed of the amphiphilic diblock copolymer PEGPCL, via oil-in-water emulsions (FIGS. 16A, 16B, 16C). The nanoparticles were characterized by UV-absorption spectroscopy and dynamic light scattering. The photothermal heating potential and photothermal stability of nanoparticles were assessed by irradiating samples using an 808 nm laser for 10 minutes at 0.75 W. Photoacoustic imaging signal was assessed by loading micelles into a phantom submerged in water. The top 5 naphthalocyanines and top 4 phthalocyanines, based on PA and heating properties, were used for in vitro and in vivo studies. The cytotoxicity and photocytotoxicity of the various micelles were assessed with ovarian cancer ID8-luciferase cells and breast cancer 4T1 cells using an MTS cell proliferation assay.

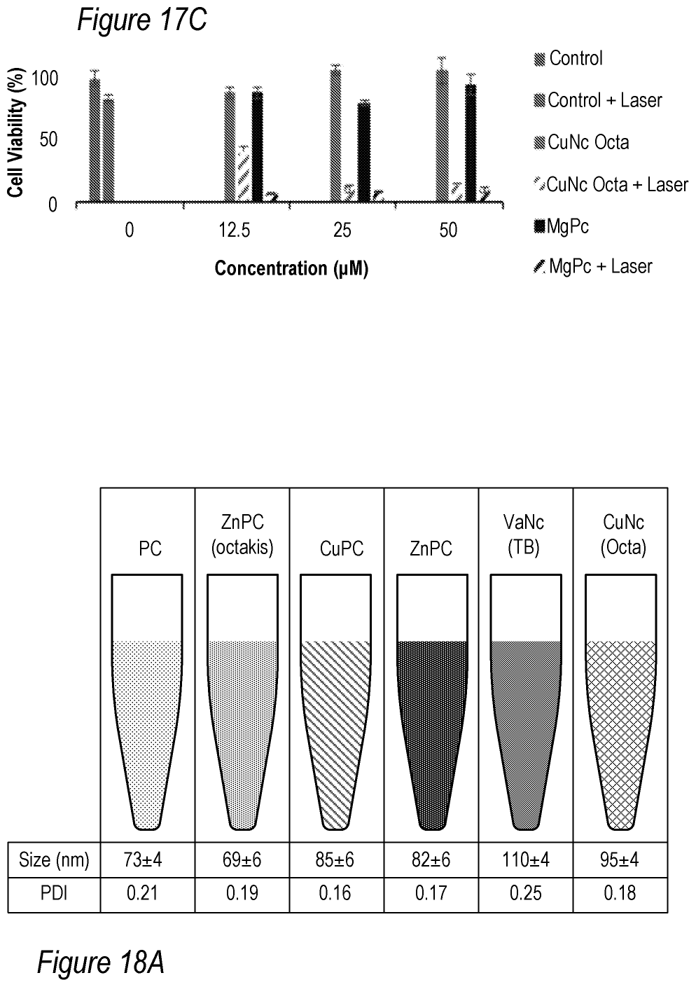

Nc—and Pc-loaded micelles demonstrated UV-absorption spectroscopy peaks ranging from 810 to 863 nm (FIG. 16D). Irradiation of Nc—and Pc-loaded micelles with an 808 nm laser caused a significant increase in solution temperature, with temperatures increasing by as much as 42.8° C., reaching a final temperature as high as 59.7° C., within 10 minutes. After three heating and cooling cycles, the Nc—and Pc-loaded micelles retained their heating ability, unlike the clinically used indocyanine green (ICG) dye (FIG. 17A). Photoacoustic imaging showed that Pc and Nc-loaded micelles are able to create strong PA signals (FIG. 17B). No toxicity was observed upon incubation of micelles with 4T1 and ID8-Luciferase cells up to dye concentrations of 50 μM; however, irradiation of these samples with an 808 nm laser led to a significant decrease in cell viability (FIG. 17C).

### Conclusion

Pc—and Nc-loaded micelles that exhibited photocytotoxicity and enhanced the photoacoustic signal were successfully prepared, making them good candidates for image-guided surgery and photothermal therapy. It is envisioned that these micelles could improve the likelihood of complete tumor removal and reduce tumor recurrence.

### Example 7

### Evaluate Pc/Nc-loaded micelles in vivo by intraoperative optical imaging and treatment

Solid tumors are often treated by surgery followed by post-surgical chemo—and radiotherapies. However, real-time intraoperative localization of the tumor margins remains a challenging task and the presence of any residual tumor left within the resected regions can result in tumor recurrence. Thus, developing a sensitive/specific image-guided resection strategy combined with local treatment could further improve the ability to eliminate residual disease. Photoacoustic imaging has been gaining clinical acceptance over the last few years and has been used for the detection of various tumor types (e.g., colon, melanoma, breast). The photoacoustic signal can be visualized in milliseconds, which is advantageous over other emerging imaging techniques, such as Raman spectroscopy and Optical Coherence Tomography, which require more time to visualize the same field of view. In addition, in comparison to fluorescence imaging, photoacoustic imaging has improved sensitivity (˜4 times) for tumors deeper than 2 mm, the ability to image deeper seated tumors (5-6 cm vs. 1 cm), and higher spatial resolution (˜100 μm).

Clinical studies have also shown that overall survival can be largely improved in patients if treated with intraoperative phototherapy as an adjunct to surgery. Previous studies also showed the benefits of combining image-guided surgery and phototherapy. Phthalocyanine (Pc) and naphthalocyanine (Nc) compounds are organic dyes that exhibit good photothermal conversion efficiency and are capable of producing both a photodynamic therapy (PDT) and photothermal therapy (PTT) effect. The major drawbacks of Pc and Nc derivatives are their poor solubility, which limits the dose, and their low molecule weight, which results in rapid clearance and poor tumor accumulation.

### Nanoparticle-Assisted Optical Imaging and Treatment

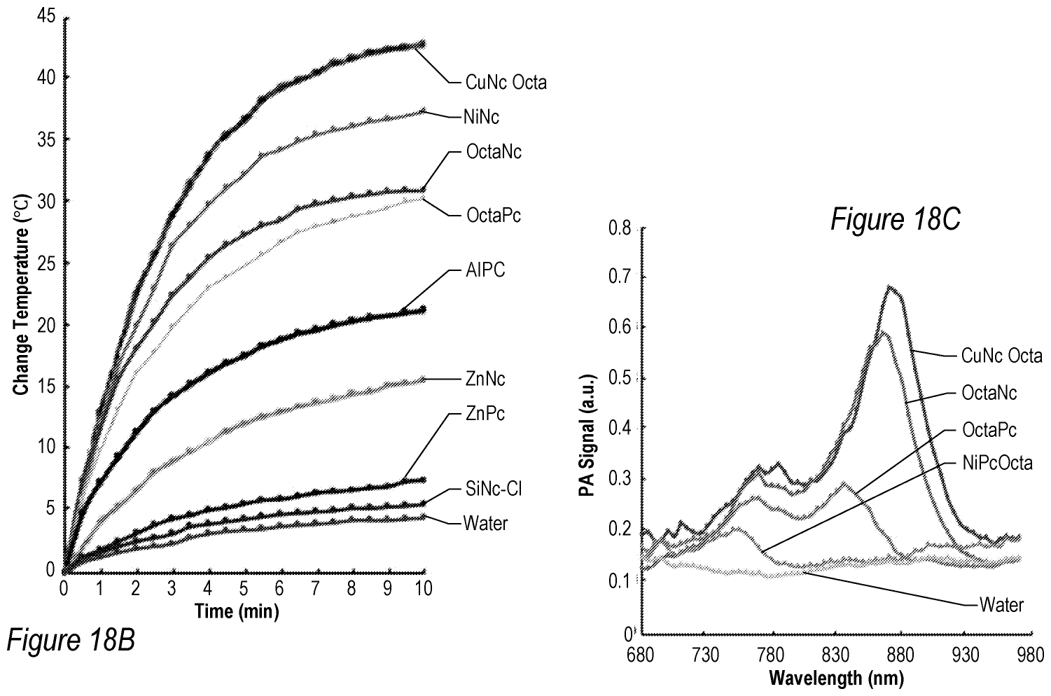

Dye-loaded micelles (FIGS. 1A and 18A) allow for the selective delivery of dyes into tumors while minimizing the uptake in normal tissue by the enhanced permeability and retention (EPR) effect. New theranostic agents for photoacoustic imaging and phototherapy that utilizes Pc and Nc dyes will be developed. Due to the high absorbance of Pc and Nc dyes at long wavelengths (λₘₐₓ>660 nm, εmax>105 Lmol−1 cm−1), and their strong photoacoustic activity (e.g., 100-fold greater than gold nanorods), their encapsulation into nanoparticles make them a favorable choice compared with other optical contrast agents such as the commonly used indocyanine green (ICG) and methylene blue (MB) dyes, which are cleared within minutes upon injection. It is believed that Pc and Nc-loaded micelles will improve the identification of tumor margins and metastases, thus improving the likelihood of a complete resection, and provide an additional PDT/PTT treatment option for the elimination of any residual disease. In preliminary studies, micelles with eight different Pc/Nc dyes were prepared. A temperature increase between 5° C. and 40° C. was observed for micelles loaded with various Pc/Nc dyes in water under 808 nm laser irradiation (0.75 W/cm2, 10 min) (FIG. 18B). The significant differences in heating observed for the small number of dyes tested, combined with the identification of a previously untested Nc dye, CuNc Octa, that outperforms all previously tested Pc/Nc dyes for PTT, encouraged us to expand the size of our library for screening, which is an important part of this proposal. Typically, an increase of just 5° C. for 10 min is sufficient for cell killing by PTT. Photoacoustic spectra indicated PA signal generation from short to long wavelengths for the various Pc/Nc-micelles (FIG. 18C). In addition, mouse-bearing breast 4T1-Luc2 flank tumors showed a strong PA signal after 24 hr post-injection of Pc-loaded micelles (FIG. 1D). In general, a positive correlation was observed between heating and photoacoustic signal, which suggests a single optimal agent is likely to perform favorably for both PTT and PAL. Therefore, it is believed Pc/Nc-loaded micelles can serve as an effective theranostic agent.

### Research Plan

Aim 1. Synthesize Pc/Nc-Loaded Polymeric Micelles and Characterize their Physical-Chemical Properties.

Fifty commercially available hydrophobic derivatives of Pc and Nc will be loaded into polymeric micelles, composed of the amphiphilic diblock copolymer PEG-PCL, via oil-in-water emulsions and their physical-chemical properties will be characterized. The structural integrity of Pc/Nc-micelles will be determined by transmission electron microscopy. The hydrodynamic diameter and zeta potential of Pc/Nc loaded polymeric micelles will be measured by dynamic light scattering. The stability and Pc/Nc-release of polymeric micelles will also be evaluated as a function of time and pH in both storage and physiologic media. The photothermal conversion coefficient and reactive oxygen species (ROS) generation will be investigated under light irradiation. Photoacoustic signal generation will be measured via phantom studies.

### Aim 2. Evaluate Pc/Nc-Loaded Micelle In Vitro.

Photocytotoxicity will be determined as a function of Pc/Nc-loaded micelle dose, as well as laser power, exposure time, and frequency of exposure. An 808 nm laser will be used for all cell studies since it is already widely utilized in a clinical setting for photothermal ablation. Cytotoxicity studies will be performed on breast cancer cells 4T1 and quantified via a cell proliferation assay (MTT). Statistical significance between groups will be determined by analysis of variance (ANOVA) or a Student's t-test where appropriate. A p<0.05 will be considered statistically significant.

### Aim 3. Evaluate Pc/Nc-Loaded Micelles In Vivo by Intraoperative Optical Imaging and Treatment.

The top five micelle formulations identified from studies in Aims 1 and 2, based on high stability, low leakage, low cytotoxicity (in the absence of laser illumination), high photoacoustic signal, and high phototoxicity, will be tested in a limited number of mice for their intratumoral accumulation and photothermal properties. It was previously found that micelles formed from the same polymer but with different dyes/drugs can exhibit significantly different pharmacokinetics and tumor accumulation. The micelle formulation that exhibits the highest accumulation, based on PAI signal intensity, and heating based on photothermal camera measurements, will be selected for additional therapeutic studies. In particular, a pilot study will be performed to examine the efficacy of PTT with the optimal Pc/Nc-micelle in a partial tumor resection model. PA imaging will be run immediately prior to and 24 hr post-intravenous injection of Pc/Nc-micelles in mice bearing 4T1-Luc2 flank tumors to assess micelle accumulation within the tumor. Firefly luciferase will allow tumor cells to be detected in live animals for recurrence studies. A partial resection will then be performed, and the surgical cavity will be exposed to an 808 nm laser to eliminate any residual cancer cells. Tumor volume and survival will be monitored as a function of time, post-treatment for up to 30 days. A log-rank analysis will be performed on Kaplan-Meier curves (generated from survival and recurrence data) to identify statistical significance (p<0.05) between groups.

Summary of Anticipated Results and their Utility for Long-Term Goals

Anticipated results: 1) Prepare reproducible, stable, and biodegradable Pc/Nc-loaded micelles with high loading capacity (>85% efficiency), low leakage (<10% in 24 hours in serum), and narrow-size distribution (PDI<0.2). 2) Demonstrate that Pc/Nc-loaded micelles can detect solid tumors (e.g., breast cancer) with high accuracy. 3) Demonstrate that Pc/Nc-loaded micelles can eliminate residual tumors via PTT/PDT. The long-term goal is to develop a new nano-based theranostic agent for more effective identification of tumor margins and metastases and treatment of residual invasive disease. It is believed that this innovative nanotechnology will profoundly improve the ability of physicians to completely eradicate the local disease in cancer patients, resulting in fewer re-excisions and ultimately leading to an improvement in progression-free and overall survival.

### Example 8

### Phthalocyanines and Napthalocyanines for

### Photoacoustic-Guided Surgery and Photothermal Therapy

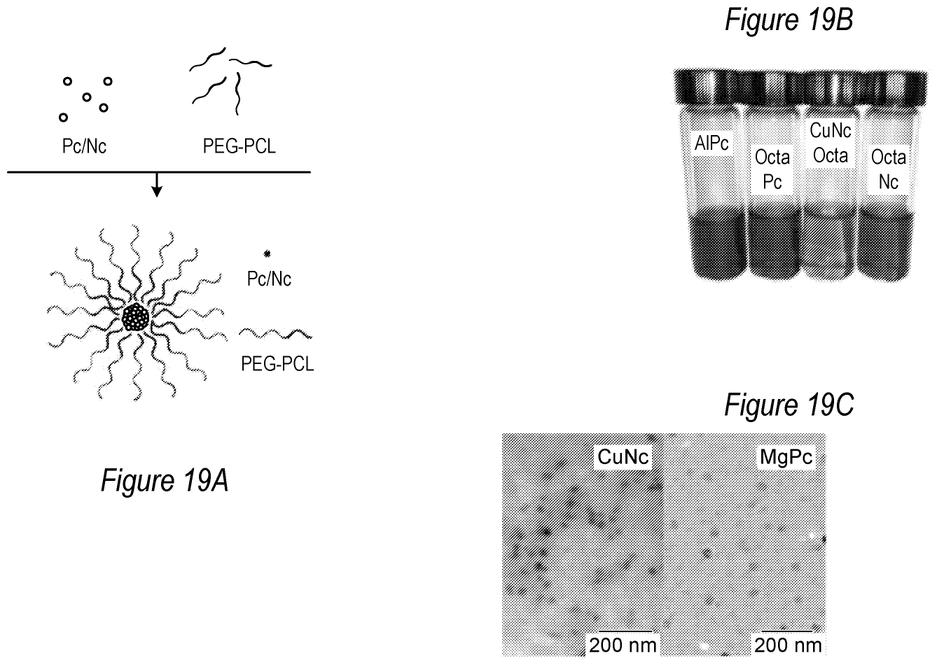

Pc/Nc-loaded micelles were formed, as shown in FIGS. 19A-19C Pc and/or Nc and the diblock copolymer PEG-PCL were first dissolved in organic solvent(s). Upon addition to water an oil-in-water emulsion was produced, whereby micelles were formed with the Pc and/or Nc encapsulated within the hydrophobic core of the polymeric micelles. (FIG. 19A); Representative photographs of Pc/Nc-loaded micelles, which were prepared using different Pc/Nc dyes (FIG. 19B); Transmission electron microscopy images of micelles loaded with CuNc Octa or MgPc (FIG. 19C).

Over fifty different PC and NC dye were screened for their photothermal properties, after being encapsulated in micelles. Several PC and NC dye were identified with exquisite photothermal and photoacoustic properties, including CuNC, NiNC, VaNC TB, MgPC, and OctaNC-loaded micelles. Preparation of the micelles is scalable. Moreover, the micelles are expected to be safe since other PC and NC dyes have been found to be safe in humans.

List of Phthalocyanine (Pc) chemicals that were loaded into micelles and evaluated for their photothermal and photoacoustic properties.

Phthalocyanines (Pcs)

| | | |
|---|---|---|
| ZnPc Octa (N/A) | ZnPc (97%) | NiPc (85%) |
| CuPc Octa (95%) | VaPc (>90%) | InPc (95%) |
| NiPc Octa (97%) | AlPc Tetra (90%) | TiOPc (>99%) |
| OctaPc (95%) | MnPc (N/A) | CoPc (97%) |
| VaPc Tetra (98%) | MgPc (90%) | CuPc (>99%) |
| PbPc Tetra (90%) | DiLiPc (70%) | PC (98%) |
| GaPc (97%) | ZnPc TB (96%) | |
| TBPc (97%) | FePc (90%) | |
| SiPc TB-OH (80%) | AlPc (85%) | |
| ZnPc Octakis (95%) | SiPc-OH (75%) | |
| Octakis Pc (N/A) | SiPc-Cl (85%) | |

List of Napthalocyanine (Nc) chemicals that were loaded into micelles and evaluated for their photothermal and photoacoustic properties.

Napthalocyanines (Ncs)

| | | |
|---|---|---|
| OctaNc (95%) | VaNc TB (95%) | NiNc (98%) |
| ZnNc (90%) | Nc (95%) | SiNc-Cl (85%) |
| CuNc Octa (90%) | CoNc (85%) | CuNc (85%) |

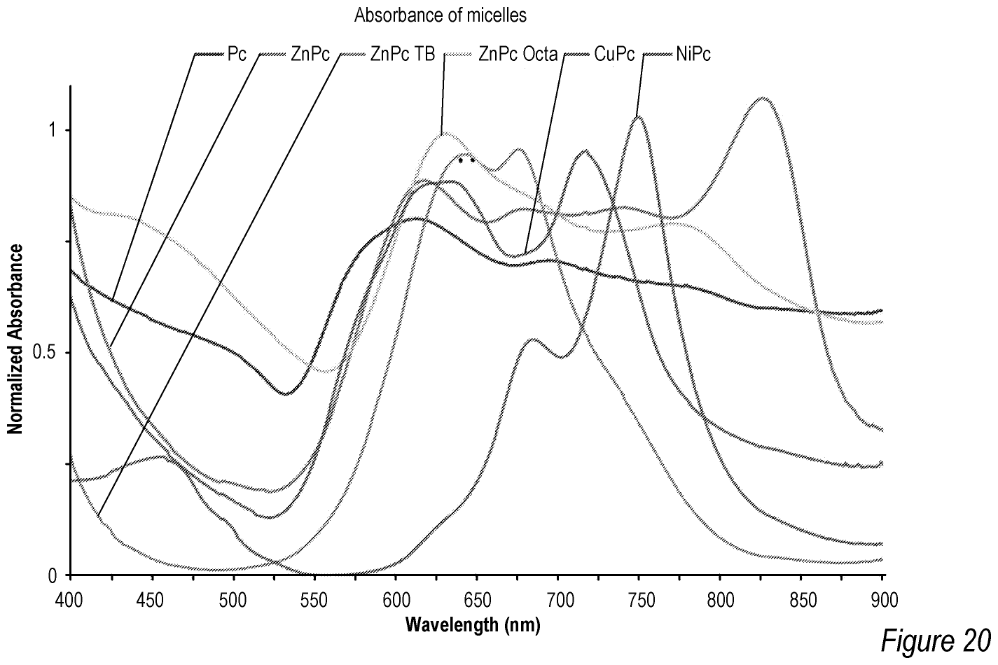

The UV absorbance spectra of six different micelles formulations, each loaded with a different Pc dye, is shown in FIG. 20.

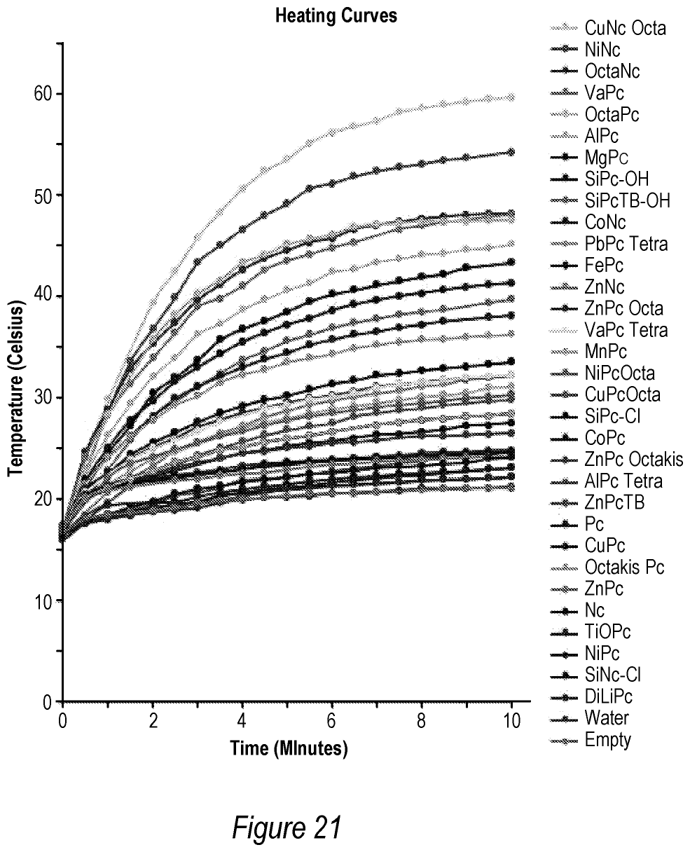

Pc and Nc-loaded micelles (9.4 uM dye concentration) and controls (empty micelles and water) were prepared and then irradiated at 808 nm (0.75 W/cm2) continuously for 10 min; heating of each sample was measured as a function of time. (FIG. 21)

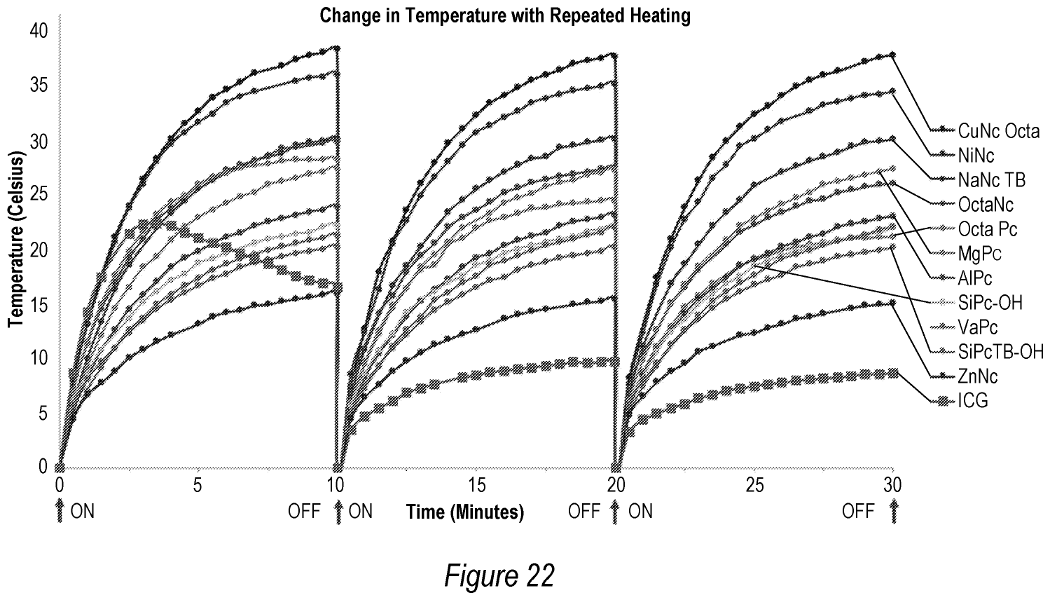

Various Pc and Nc-loaded micelles (9.4 uM) were heated multiple times, using a laser (0.75 W/cm2), in 10-min increments. (FIG. 22) The samples were allowed to cool to room temperature between successive rounds. While ICG lost its ability to heat upon laser irradiation, the Pc and Nc-dye loaded micelles exhibited excellent stability, with little to no loss in the ability to generate heat even after 30 minutes of total irradiation.

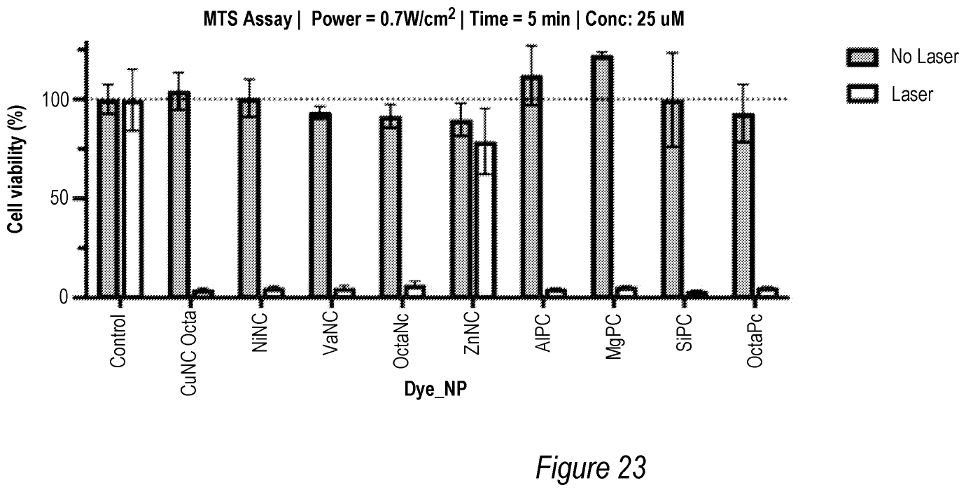

FIG. 23 shows an MTS cell proliferation assay of 4T1 cells after incubation with Nc or Pc-loaded micelles or standard media for 24 h. No significant toxicity was observed in the absence of laser irradiation; however, upon laser irradiation at 808 nm (0.75 W/cm2) for 5 min, each micelle formulation tested led to near complete cell killing, with the exception of ZnNc-loaded micelles, which exhibited very little cell killing.

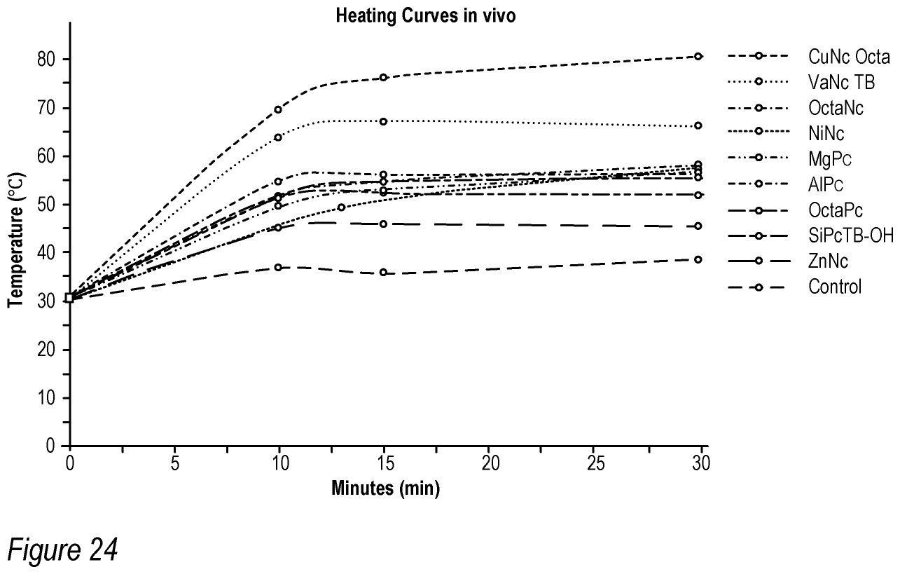

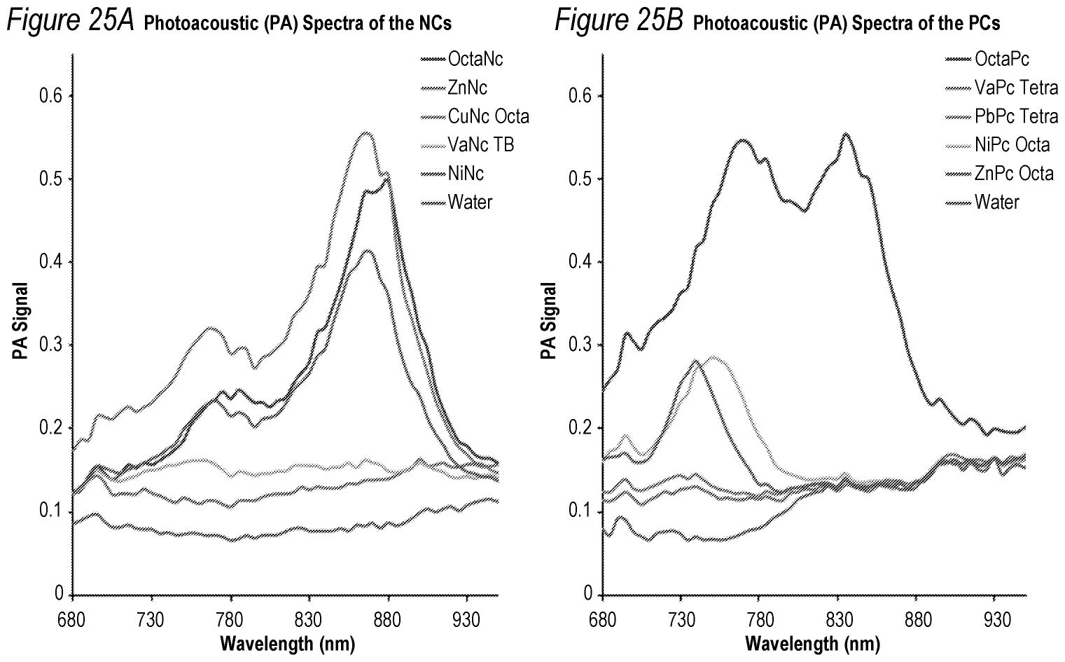

4T1-tumor bearing mice had various Pc or Nc-loaded micelles administered intravenously (10 mg/kg). (FIG. 24) After 24 hours, the tumors were irradiated with a laser (808 nm, 0.75W) for 30 min. The change in tumor temperature was monitored via thermal imaging as a function of time. Photoacoustic spectra of various Nc-(A) and Pc-loaded (B) micelle formulations are shown in FIGS. 25A-25B.

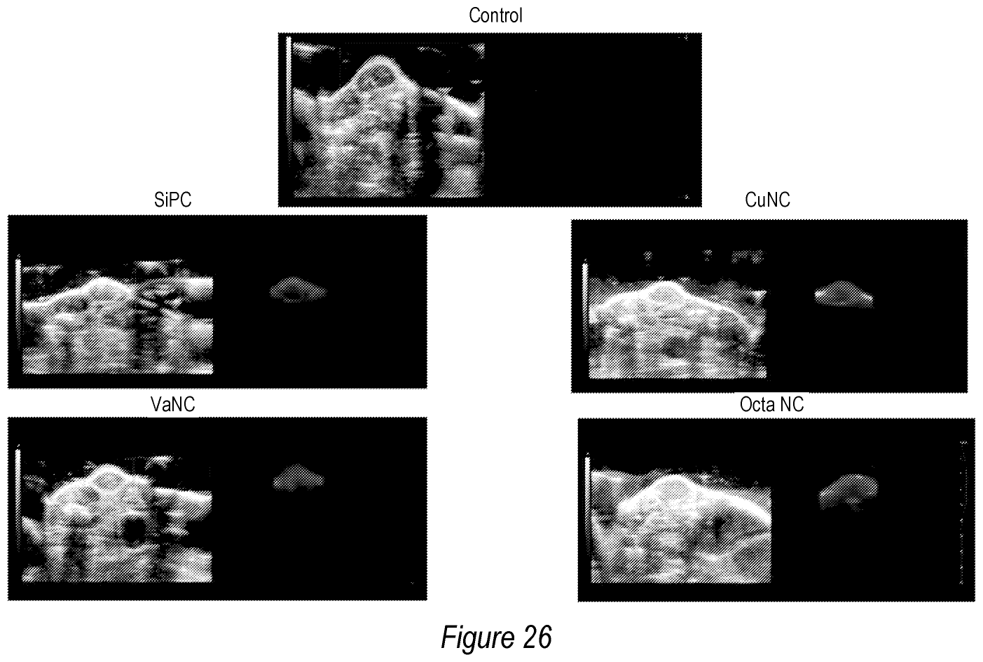

Representative photoacoustic images of 4T1 flank tumors in mice 24 hours after intravenous administration of SiPC-, CuNC-, VaNC-, and Octa NC-loaded micelles are shown in FIG. 26. Control animals did not receive any contrast agents. Left, ultrasound image; right, photoacoustic (color) images.

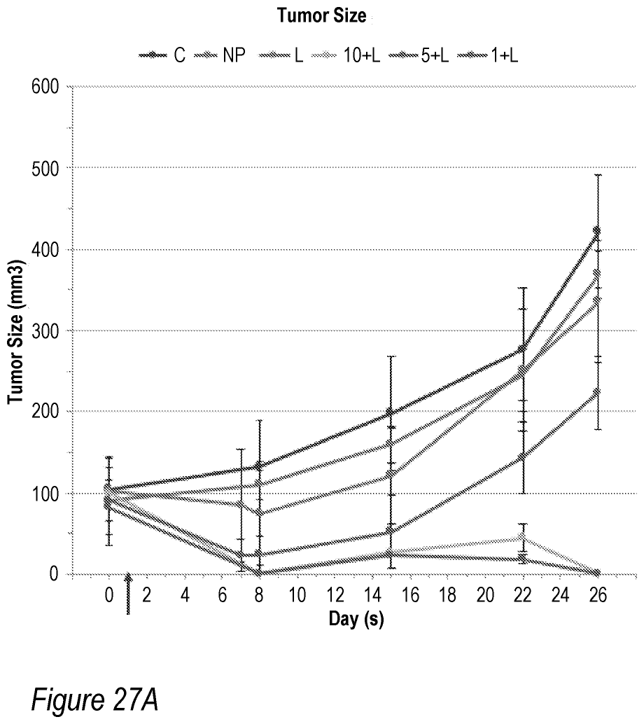

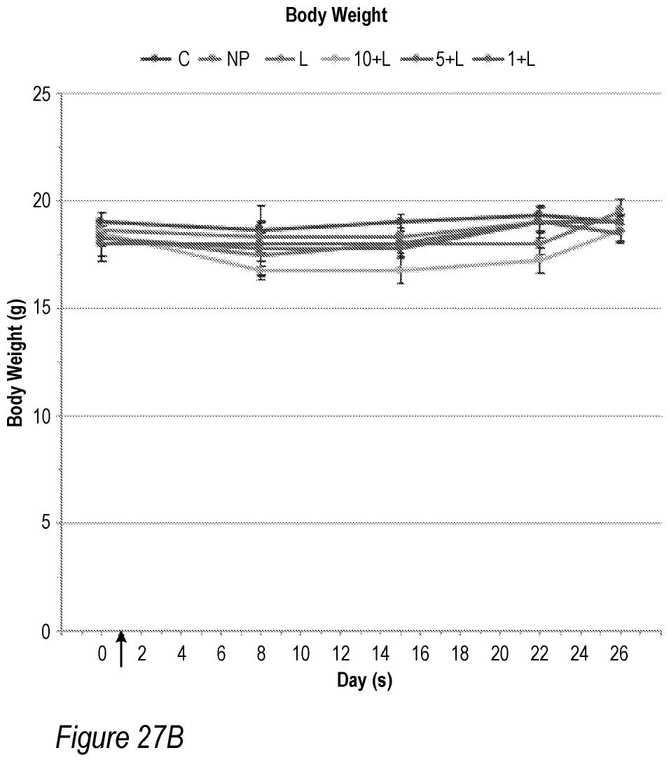

Mice bearing 4T1 breast flank tumors were injected with saline, CuNc Octa-loaded micelles (NP, 10 mg/kg, no laser), or CuNc Octa (10, 5, 1 mg/kg, with laser) (n=5 per group). (FIGS. 27A-27B) The subset of mice that received subcutaneous laser irradiation (0.7 W/cm2, 10 min) received this laser treatment 24 hrs following the injection of the micelles. The tumor size of all mice was monitored as a function of time (FIG. 27A). The body weight of all mice was also monitored as a function of time (FIG. 27B). The results demonstrate PTT efficacy and safety using CuNc Octa micelles according to embodiments of the invention.

All references, including publications, patent applications, and patents, cited herein are hereby incorporated by reference in its entirety herein.

Having described preferred embodiments of the invention with reference to the accompanying drawings, it is to be understood that the invention is not limited to the precise embodiments, and that various changes and modifications may be affected therein by those skilled in the art without departing from the scope or spirit of the invention as defined in the appended claims.

What is claimed is:

1. A dye-loaded nanoparticle comprising a phthahalocyanine (PC) dye, naphthalocyanine (NC) dye, or combination thereof solubilized within a micelle, polymersome, or liposome.

2. The dye-loaded nanoparticle of claim 1, wherein the PC dye further includes octabutoxy, hexadecafluoro, tetrakis(4-cumylphenoxy), tetra-tert-butyl, tetraphenoxy, tetrakis(phenylthio) or octakis(octyloxy).

3. The PC dye of claim 1, wherein the PC dye is without or with a metal center and wherein the metal center includes zinc, nickel, copper, iron, magnesium, aluminum, gallium, dilithium, titanyl, vanadyl, cobalt, tin, titanium, manganese, indium or lead

4. The dye-loaded nanoparticle of claim 1, wherein the NC dye further includes octabutoxy, bis(trihexylsilyloxide) or tetra-tert-butyl.

5. The NC dye of claim 1, wherein the NC dye is without or with a metal center and wherein the metal center includes zinc, nickel, copper, iron, magnesium, aluminum, gallium, silicon, dilithium, titanyl, silicon, vanadyl, cobalt, tin, titanium, manganese, indium or lead.

6. The dye-loaded nanoparticle of claim 1, wherein the PC dye is Zinc phthalocyanine (ZnPC).

7. The dye-loaded nanoparticle of claim 1, wherein the NC dye is Zinc naphthalocyanine (ZnNC).

8. The dye-loaded nanoparticle of claim 1, wherein the dye is a mixture of Zinc phthalocyanine and Zinc naphthalocyanine (ZnPC-NC).

9. The dye-loaded nanoparticle of claim 1, wherein the nanoparticle comprises an amphiphilic polymer or lipid or combination thereof.

10. The dye-loaded nanoparticle of claim 9, wherein the amphiphilic polymer is a diblock copolymer, and includes poly(ethylene glycol)-poly(caprolactone) (PEG-PCL), poly(ethylene glycol)-poly(lactide-co-glycolide) (PEG-PLGA), poly(ethylene glycol)-poly(lactic acid) (PEG-PLA), poly(ethylene glycol)-poly(β-benzyl-L-aspartate) (PEG-PBLA), or poly(ethylene glycol)-poly(amino acid).

11. The dye-loaded nanoparticle of claim 9, wherein the lipid is a Pluronic, 1,2-dipalmitoyl-sn-glycero-3-phosphocholine (DPPC), L-α-phosphatidylcholine hydrogenated (HSPC), distearoyl phosphatidyl ethanolamine-polyethylene glycol (DPSE-PEG), or L-6-phosphatidylcholine (EggPC).

12. The dye-loaded nanoparticle of claim 9, wherein the dye:amphiphile ratio (w/w) is in the range of 1:10 to 6:1.

13. The dye-loaded nanoparticle of claim 6, wherein the nanoparticle is a micelle and the dye ZnPC:PEG-PCL ratio (w/w) is 1:2.

14. The dye-loaded nanoparticle of claim 1, wherein the nanoparticle is further functionalized with a targeting agent.

15. The dye-loaded nanoparticle of claim 14, wherein the targeting agent is an antibody, antibody fragments, affibody, DARPin, nanobody, protein, peptide, aptamer, or small molecule.

16. A method for preparing a dye-loaded nanoparticle, the method comprising:
a) solubilizing a phthalocyanine (PC) dye, an naphthalocyanine (NC) dye, or combination thereof, within an amphiphilic polymer or a lipid to form dye-loaded nanparticle micelles, polymersomes, or liposomes; and
b) purifying the PC-loaded nanoparticulate micelles by dialysis, size exclusion chromatography, filtration, or any combination thereof.

17. The method of claim 16 wherein micelles are formed in an oil-in-water emulsion

18. The method of claim 17 wherein the dye, polymer, lipid, or combination thereof are dissolved in the oil phase.

19. The method of claim 16, wherein the PC dye further includes octabutoxy, hexadecafluoro, tetrakis(4-cumylphenoxy), tetra-tert-butyl, tetraphenoxy, tetrakis(phenylthio) or octakis(octyloxy).

20. The method of claim 16, wherein the PC dye is without or with a metal center and wherein the metal center includes zinc, nickel, copper, iron, magnesium, aluminum, gallium, silicon, dilithium, titanyl, vanadyl, cobalt, tin, titanium, manganese, indium or lead.

21. The method of claim 16, wherein the NC dye further includes octabutoxy, bis(trihexylsilyloxide) or tetra-tert-butyl.

22. The method of claim 16, wherein the NC dye is without or with a metal center and wherein the metal center includes zinc, nickel, copper, gallium, silicon, vanadyl, cobalt, tin, titanium, manganese, indium or lead.

23. The method of claim 16, wherein the PC dye is Zinc phthalocyanine (ZnPC).

24. The method of claim 16, wherein the NC dye is Zinc naphthalocyanine (ZnNC).

25. The method of claim 16, wherein the dye is a mixture of Zinc phthalocyanine and Zinc naphthalocyanine (ZnPC-NC).

26. The method of claim 16, wherein the nanoparticle comprises an amphiphilic polymer or lipid or combination thereof.

27. The method of claim 26, wherein the amphiphilic polymer is a diblock copolymer, and includes poly(ethylene glycol)-poly(caprolactone) (PEG-PCL), poly(ethylene glycol)-poly(lactide-co-glycolide) (PEG-PLGA), poly(ethylene glycol)-poly(lactic acid) (PEG-PL A), poly(ethylene glycol)-poly(β-benzyl-L-aspartate) (PEG-PBLA), or poly(ethylene glycol)-poly (amino acid).

28. The method of claim 26, wherein the lipid is a Pluronic, 1,2-dipalmitoyl-sn-glycero-3-phosphocholine (DPPC), L-α-phosphatidylcholine hydrogenated (HSPC), distearoyl phosphatidyl ethanolamine-polyethylene glycol (DSPE-PEG), or L-α-phosphatidylcholine (EGGPC).

29. The method of claim 26, wherein the dye:amphiphile ratio (w/w) is in the range of 1:10 to 6:1.

30. The method of claim 26, wherein the nanoparticle is a micelle and the dye ZnPC:PEG-PCL ratio (w/w) is 1:2.

31. A method for visualization of a tumor without optical imaging equipment, the method comprising:
a) injecting a subject with dye-loaded nanoparticles; and
b) detecting by direct visualization, camera, or endoscopy an accumulation of colored dye in a tumor and a demarcation of tumor margins.

32. A method for visualization of a tumor with photoacoustic imaging equipment, the method comprising:
a) injecting a subject with dye-loaded nanoparticles; and
b) detecting by photoacoustic imaging an accumulation of dye in a tumor and a demarcation of tumor margins.

33. The method of any one of claims 31 to 32, wherein the dye-loaded nanoparticles are injected at a dye dose in the range of 1 mg/kg to 50 mg/kg.

34. The method of any one of claims 31 to 32, wherein the dye is visualized or detected one day after injection of the dye-loaded nanoparticles.

35. The method of any one of claims 31 to 32, wherein the tumor is a nonpolypoid or polypoid colorectal adenoma.

36. The method of any one of claims 31 to 32, further comprising resecting the tumor within a 1 cm radius of the tumor margins.

37. The method of any one of claims 31 to 32, wherein the PC dye is Zinc 2,3,9,10,16,17,23,24-octakis(octyloxy)-29H,31H-phthalocyanine (ZnPC(octakis)), Nickel(II) 1,4,8,11,15,18,22,25-octabutoxy-29H,31H-phthalocyanine (NiPC), Copper(II) phthalocyanine (CuPC), Zinc phthalocyanine (ZnPC) or Zinc 2,9,16,23-tetra-tert-butyl-29H,31H-phthalocyanine (ZnPC(TB)).

38. The method of any one of claims 31 to 32, wherein the PC dye is Zinc phthalocyanine (ZnPC).

39. The method of any one of claims 31 to 32, wherein the nanoparticle comprises an amphiphilic polymer or lipid or combination thereof, and the amphiphilic polymer is diblock copolymer (ethylene glycol)-co-poly(caprolactone) (PEG-PCL).

40. The method of any one of claims 31 to 32, wherein the PC dye Zn(TB):PEG-PCL ratio (w/w) is in the range of 1:4 to 1:1.

41. The method of claim 40, wherein the PC dye Zn(TB):PEG-PCL ratio (w/w) is 1:2.

42. A method for detecting colorectal cancer, the method comprising:
a) injecting a subject with phthalocyanine (PC)-loaded nanoparticulate amphiphilic polymer or lipid micelles; and
b) detecting by endoscopy an accumulation of blue dye in a tumor and a demarcation of tumor margins.

43. The method of claim 42, wherein the phthalocyanine (PC)-loaded nanoparticulate polymer or lipid micelles are injected at a injected at a dye dose in the range of 1 mg/kg to 50 mg/kg.

44. The method of claim 42, wherein the dye is visualized or detected one day after injection of the dye-loaded nanoparticles.

45. The method of claim 42, wherein the tumor is a nonpolypoid or polypoid colorectal adenoma.

46. The method of claim 42, further comprising resecting the tumor within a 1 cm radius of the tumor margins.

47. The method of claim 42, wherein the PC dye is Zinc 2,3,9,10,16,17,23,24-octakis(octyloxy)-29H,31H-phthalocyanine (ZnPC(octakis)), Nickel(II) 1,4,8,11,15,18,22,25-octabutoxy-29H,31H-phthalocyanine (NiPC), Copper(II) phthalocyanine (CuPC), Zinc phthalocyanine (ZnPC) or Zinc 2,9,16,23-tetra-tert-butyl-29H,31H-phthalocyanine (ZnPC(TB)).

48. The method of claim 42, wherein the PC dye is Zinc phthalocyanine (ZnPC).

49. The method of claim 42, wherein the amphiphilic polymer is diblock copolymer (ethylene glycol)-co-poly(caprolactone) (PEG-PCL).

50. The method of claim 42, wherein the PC dye Zn(TB):PEG-PCL ratio (w/w) is in the range of 1:4 to 1:1.

51. The method of claim 42, wherein the PC dye Zn(TB):PEG-PCL ratio (w/w) is 1:2.

52. A method for detecting ovarian or breast cancer, the method comprising:
a) injecting a subject with phthalocyanine (PC)-loaded nanoparticulate amphiphilic polymer or lipid micelles; and
b) detecting by intraoperative photoacoustic imaging an accumulation of blue dye in a tumor and a demarcation of tumor margins.

53. The method of claim 52, wherein the dye-loaded nanoparticles are injected at a dye dose in the range of 1 mg/kg to 50 mg/kg.

54. The method of claim 52, wherein the dye is visualized or detected one day after injection of the dye-loaded nanoparticles.

55. A method for treating a tumor, the method comprising:
a) administering intravenously to a subject phthalocyanine (PC) dye-loaded or naphthalocyanine (NC) dye-loaded nanoparticulate amphiphilic polymer or lipid micelles;
b) detecting by intraoperative photoacoustic imaging an accumulation of dye in a tumor and a demarcation of tumor margins; and/or
c) irradiating the PC dye or the NC dye within the tumor with a laser to heat the tumor, thereby decreasing tumor cell viability and/or killing the tumor cells.

56. The method of claim 55, wherein the PC dye is ZnPc Octa, CuPc Octa, NiPc Octa, OctaPc, VaPc Tetra, PbPc Tetra, GaPc, TBPc, SiPc TB-OH, ZnPc Octakis, Octakis Pc, ZnPc, VaPc, AlPc Tetra, MnPc, MgPc, DiLiPc, ZnPc TB, FePc, AlPc, SiPc-OH, SiPc-Cl, NiPc, InPc, TiOPc, CoPc or CuPc.

57. The method of claim 55, wherein the PC dye is MgPC, VaPC, OctaPC or A1PC.

58. The method of claim 55, wherein the NC dye is OctaNc, ZnNc, CuNc Octa, VaNc TB, Nc CoNc, NiNc, SiNc-Cl or CuNc.

59. The method of claim 55, wherein the NC dye is CuNC, NiNC, VaNC TB, and OctaNC.

60. The method of claim 55, wherein the detecting by intraoperative photoacoustic imaging localize to the tumor and assess the entire margin and the irradiating are in real-time.

61. The method of claim 55, wherein the tumor is heated to 39° C. to 70° C. within 10 minutes.

62. The method of claim 55, wherein the tumor is an ovarian tumor or a breast cancer tumor.

63. The method of claim 55, wherein the tumor is a nonpolypoid or polypoid colorectal adenoma.

64. The method of claim 55, wherein the tumor is a colorectal cancer.

65. The method of claim 55, wherein the PC or NC dye is visualized or detected one day after intravenous administration of the dye-loaded nanoparticulate amphiphilic polymer or lipid micelles.

66. The method of claim 55, wherein dye-loaded nanoparticulate comprises an amphiphilic polymer or lipid or combination thereof.

67. The method of claim 55, wherein the amphiphilic polymer is a diblock copolymer, and includes poly(ethylene glycol)-poly(caprolactone) (PEG-PCL), poly(ethylene glycol)-poly(lactide-co-glycolide) (PEG-PLGA), poly(ethylene glycol)-poly(lactic acid) (PEG-PLA), poly(ethylene glycol)-poly(β-benzyl-L-aspartate) (PEG-PBLA), or poly(ethylene glycol)-poly(amino acid).

68. The method of claim 55, wherein the lipid is a Pluronic, 1,2-dipalmitoyl-sn-glycero-3-phosphocholine (DPPC), L-α-phosphatidylcholine hydrogenated (HSPC), distearoyl phosphatidyl ethanolamine-polyethylene glycol (DPSE-PEG), or L-α-phosphatidylcholine (EGGPC).

69. The method of claim 55, wherein the wavelength of the laser is near the peak absorbance of the PC dye or the NC dye.

70. The method of claim 69, wherein the wavelength of the laser is 808 nm.
